(19) United States
(12) Patent Application Publication
(10) Pub. No.: US 2019/0282701 A1
(43) Pub. Date: Sep. 19, 2019

(54) CONJUGATES OF HYALURONIC ACID AND ANTICANCER COMPOUNDS

(71) Applicant: THE UNIVERSITY OF MANCHESTER, Manchester (GB)

(72) Inventors: Nicola TIRELLI, Manchester (GB); Vincenzo QUAGLIARIELLO, Caserta (IT); Alfonso BARBARISI, Caserta (IT); Francesco ROSSO, Caserta (IT); Som Akshay JAIN, Caserta (IT); Manlio BARBARISI, Caserta (IT); Rosario Vincenzo LAFFAIOLI, Napoli (IT); Ian James STRATFORD, Manchester (GB); Muna OQAL, Manchester (GB); Manal MEHIBEL, Manchester (GB)

(21) Appl. No.: 16/318,328

(22) PCT Filed: Jul. 25, 2017
(86) PCT No.: PCT/GB2017/052174

§ 371 Date: Jan. 16, 2019

(30) Foreign Application Priority Data
Jul. 25, 2016 (GB) 1612872.0

(51) Int. Cl. A61K 47/61; A61K 31/353; A61P 35/00

(52) U.S. Cl. CPC A61K 47/61; A61P 35/00; A61K 31/353

(57) ABSTRACT

The present invention relates to a polymer-drug conjugate wherein the polymer is hyaluronic acid and the drug is an anticancer compound. The anticancer compound is covalently linked to the hyaluronic acid by a pH-labile boronic acid-containing linkage. These conjugates can be used for the treatment of cancer.

### INTRODUCTION

The present invention relates to prodrug compounds. More specifically, the present invention relates to certain hyaluronic acid polymer-anticancer drug conjugates that function as prodrugs. These conjugates can be used for the treatment of cancer.

### BACKGROUND OF THE INVENTION

Cancer is caused by uncontrolled and unregulated cellular proliferation. Precisely what causes a cell to become malignant and proliferate in an uncontrolled and unregulated manner has been the focus of intense research over recent decades.

One key challenge that remains with modern day cancer therapy is the identification of effective strategies for selectively targeting the potent anticancer drug compounds that are available to the tumour site, thereby minimising the exposure of healthy body tissues to these potent, and often toxic, agents.

Numerous different formulation and prodrug strategies have been developed and trialled over the years, but there still remains a need for new approaches to selectively and effectively target tumours with potent anticancer drugs.

The present invention was devised with the foregoing in mind.

### SUMMARY OF THE INVENTION

In one aspect, the present invention provides a polymer-drug conjugate, or a pharmaceutically acceptable salt or solvate thereof, wherein the polymer is a modified hyaluronic acid derivative and the drug is an anticancer compound, and wherein the anticancer compound is covalently linked to the modified hyaluronic acid derivative by a pH-labile boron-containing linkage.

In another aspect, the present invention provides a pharmaceutical composition comprising a polymer-drug conjugate as defined herein, or a pharmaceutically acceptable salt or solvate thereof, and one or more pharmaceutically acceptable excipients.

In another aspect, the present invention provides a polymer-drug conjugate as defined herein, or a pharmaceutically acceptable salt or solvate thereof, or a pharmaceutical composition as defined herein, for use in the treatment of cancer. In a particular embodiment, the cancer is a human cancer.

In another aspect, the present invention provides a polymer-drug conjugate as defined herein, or a pharmaceutically acceptable salt or solvate thereof, or a pharmaceutical composition as defined herein, for use in the treatment of solid tumours.

In another aspect, the present invention provides the use of a polymer-drug conjugate as defined herein, or a pharmaceutically acceptable salt or solvate thereof, in the manufacture of a medicament for use in treatment of cancer. Suitably, the medicament is for use in the treatment of human cancers.

In another aspect, the present invention provides the use of a polymer-drug conjugate as defined herein, or a pharmaceutically acceptable salt or solvate thereof, in the manufacture of a medicament for use in the treatment of a solid tumour.

In another aspect, the present invention provides a method of inhibiting cell proliferation, reducing cell viability, increasing their susceptibility of cells to other antiproliferative or anticancer drugs, or any combination thereof, in vitro or in vivo, said method comprising contacting a cell with an effective amount of a polymer-drug conjugate as defined herein, or a pharmaceutically acceptable salt or solvate thereof.

In another aspect, the present invention provides a method of treating cancer in a patient in need of such treatment, said method comprising administering to said patient a therapeutically effective amount of a polymer-drug conjugate as defined herein, or a pharmaceutically acceptable salt or solvate thereof, or a pharmaceutical composition as defined herein.

In another aspect, the present invention provides a method of treating a solid tumour in a patient in need of such treatment, said method comprising administering to said patient a therapeutically effective amount of a polymer-drug conjugate as defined herein, or a pharmaceutically acceptable salt or solvate thereof, or a pharmaceutical composition as defined herein.

The present invention further provides a method of synthesising a polymer-drug conjugate as defined herein, or a pharmaceutically acceptable salt or solvate thereof.

In another aspect, the present invention provides a polymer-drug conjugate as defined herein, or a pharmaceutically acceptable salt or solvate thereof, obtainable by, or obtained by, or directly obtained by a method of synthesis as defined herein.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “anticancer drug” refers to a drug molecule showing pharmacological activity useful for the treatment of cancer.

The term “pH-labile” is used herein to refer to the cyclic boronic ester linkages or related boron-containing linkers, which are stable at normal physiological pH values (e.g. pH 7 to 8 and typically around pH 7.4) but which cleave following exposure to the mild acidic conditions, which can occur, for example, in the endosome following endocytosis, in a phagosome following phagocytosis, and/or in the interstitial spaces in tumours. By “mild acidic conditions”, we mean pH values that are typically in the range of pH 5 to 6.5, or more typically pH 5 to 6.

By “boron-containing linkage”, we mean any chemical group whose structure is based on a central tri- or tetravalent boron atom and can be summarised as:

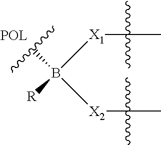

wherein

POL represents the modified HA polymer and the point of attachment is a carbon-boron bond;

X₁ and X₂ are heteroatoms selected from O, N or S; and X₁ and X₂ both connect the boron atom to the anticancer drug molecule;

R is absent or selected from: A) an OH group; or B) a group —Xᵣ-Qᵣ- formed by the association of a substiuent group of the formula -QᵣXᵣH present on either the modified HA polymer or the drug molecule with the boron atom, wherein Xᵣ is a heteroatom linker selected from —O—, —NR_{z}— (where R_{z} is H or (1-4C)alkyl) or —S— and Qᵣ is the remainder of the substituent group present on either the modified HA polymer or the drug molecule. As X₁ and X₂ provide two points of connection to the anticancer drug molecule, the boron-containing linkage forms a cyclic group and may therefore be referred to as cyclic boronic linkages. Compounds in which X₁ and X₂ are both O are known as cyclic boronic esters and compounds in which X₁ and X₂ are both N are known as cyclic boronic amides. In an embodiment, R is absent. In another embodiment, R is a —OH group, which quaternarizes the boron atom at neutral or basic pH. In another embodiment, R may be a group linked via a heteroatom Xᵣ a heteroatom (O, N, S)-terminated group connected either to the modified HA polymer or to the drug molecule; this internal ligation can be used to improve the stability of the boron-containing linkage against hydrolysis.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl or naphthyl, especially phenyl.

The term “alkylene” refers to an alkyl linker that links two or moieties together.

The term “arylene” refers to an aryl linker group (e.g. phenylene (—C₆H₄—)) that linkes two or more moieties together.

The term “optionally substituted” refers to either groups, structures, or molecules that are substituted and those that are not substituted. Where optional substituents are chosen from “one or more” groups it is to be understood that this definition includes all substituents being chosen from one of the specified groups or the substituents being chosen from two or more of the specified groups. Examples of optional substituents include halo, cyano, nitro, hydroxy, mercapto, amino, carboxy, carbamoyl, (1-6C)alkyl, (2-6C)alkenyl, (2-6C)alkynyl, (1-6C)alkoxy, (2-6C)alkenyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulphamoyl, N,N-di-[(1-6C)alkyl]sulphamoyl, and wherein any alkyl moiety in any of the aforementioned groups is optionally substituted with one or more halo, cyano, nitro, hydroxy, mercapto, amino, carboxy, carbamoyl, (1-6C)alkoxy, (1-6C)alkylthio, (1-6C)alkylsulphinyl, (1-6C)alkylsulphonyl, (1-6C)alkylamino, or di-[(1-6C)alkyl]amino.

### Polymer-Drug Conjugates (Pro-Drugs)

In one aspect, the present invention provides a polymer-drug conjugate, or a pharmaceutically acceptable salt or solvate thereof, wherein the polymer is hyaluronic acid and the drug is an anticancer compound, and wherein the anticancer compound is covalently linked to the hyaluronic acid by a pH-labile boron-containing linkage.

The polymer-drug conjugates of the present invention possess a number of important and advantageous properties.

Firstly, the macromolecular structure of the polymer-drug conjugate can confer some chemical stability to the anticancer drug molecule. In particular, the formation of the boronic ester linkages with the anticancer drug compound can protect certain functional groups present on the anticancer compounds (such as, for example, 1,2-di-hydroxybenzene groups or 1,2-diaminobenzene groups present on the drug molecule) that might otherwise be prone to chemical and/or enzymatic degradation, typically of oxidative nature. Additionally, in some cases the polymer-drug conjugate may mask the antigenicity and/or toxicity of the anticancer compound.

The macromolecular structure of the polymer-drug conjugates of the present invention may also reduce the clearance of the anticancer drug through kidneys, which is a phenomenon that is size-sensitive, with a size threshold generally accepted to be below the size of serum albumin, i.e. within the range of 2-4 nm. Additionally, the non-antigenicity of the hyaluronic acid polymer may minimise the likelihood of the polymer-drug conjugate being recognised as a foreign body and cleared from the circulation.

The macromolecular polymer-drug conjugates of the present invention will also preferentially accumulate in sites characterised by increased capillary leakiness, such as, for example, in solid tumoural tissues. The reduced lymphatic drainage in solid tumours also contributes to the final Enhanced Permeation and Retention effect (EPR effect) that allows colloids with a prolonged circulation times to preferentially localise in tumours.

The hyaluronic acid portion of the polymer-drug conjugate of the present invention is also capable of exploiting receptor-mediated endocytosis mechanisms that can further enhance tumour targeting. Hyaluronic acid can bind to a number of receptors. One of the main HA receptors is CD44, which has an endocytic function. It is believed that the polymer-drug conjugates of the present invention will be recognised and taken up into cells in a manner that is substantially proportional to the expression of CD44. Receptors for hyaluronic acid, such as CD44, are often overexpressed in tumours. Furthermore, CD44 is also considered to be a cancer stem cell marker. In particular, some CD44 variants, for example variant 6, are expressed almost exclusively in tumors. The ability of HA to bind to CD44 will enhance the localisation of the polymer-drug conjugates of the present invention at tumour sites as well as providing a means for enhancing the intracellular uptake of the drug into tumour cells.

The boron-containing linkage present in the conjugates of the present invention possess good chemical stability in an aqueous environment. In preferred embodiments, where the cyclic boronic ester linkages are formed with catechol moieties present in the anticancer drug molecule, or catechol moieties present in a linker attached group attached to the anticancer drug molecule, the cyclic boronic ester linkages are characterized by good chemical binding strength, with a Kd in the order of a few hundreds μM or lower at physiological pH. The binding strength can be further increased by increasing the number of boronic groups present on the hyaluronic acid in order to have an excess of boronic acid groups per each available catechol moiety, which provides an increase of the avidity of the hyaluronic acid derivative for the drug. The boronic ester linkages also readily dissociate when the pH declines to, for example, less than pH 6. This can allow release of the bound anticancer drug molecule in environments characterised by an acidic pH, such as the environment in the endo/phagosomes (after cellular uptake via receptor-mediated endocytosis), acidic environments in hypoxic tumour cells, or possibly also in the tumor interstitial space (extracellular release after accumulation in tumours).

### The Modified Hyaluronic Acid (HA)

Hyaluronic acid, also referred to as “HA”, is a naturally occurring, water soluble polysaccharide that is a major component of the extra-cellular matrix and is widely distributed in animal tissues. Naturally occurring HA generally has a molecular weight range of about between 6×10⁴ to about 8×10⁶ Daltons. It has excellent biocompatibility and does not give a foreign body or allergic reaction when injected into a subject. HA is also widely used as a biomaterial, in particular as a filler in cosmetic surgery and in ophthalmology, for viscosupplementation in osteoarthritis and also in several regenerative medicine scenarios.

In the drug conjugates of the present invention, the modified hyaluronic acid is a soluble HA polymer that has a molecular weight within the range of 100 kDa to 1 MDa. Suitably, the modified hyaluronic acid polymer suitably has a molecular weight within the range of 100 kDa to 800 kDa. More suitably, the modified hyaluronic acid polymer suitably has a molecular weight within the range of 300 kDa to 500 kDa.

The term “modified Hyaluronic acid polymer” or “modified HA” is used herein to refer to a hyaluronic acid polymer in which a proportion of disaccharide monomeric units have been chemically modified to incorporate pendant boronic acid-containing moieties.

A single monomeric unit of hyaluronic acid is shown below:

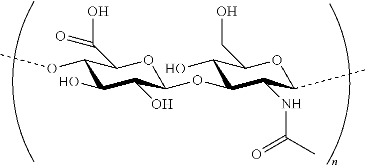

The carboxylic acid (—CO₂H) group present in the HA monomeric unit shown above provides a convenient means to couple a pendant boronic acid-containing moiety to the HA polymer backbone. A person skilled in the art will appreciate that a number of different chemical couplings are feasible at such carboxylic acid groups. For example, the reaction of an amine group present on the boronic acid-containing moiety with the carboxylic acid group present on the HA monomer will form an amide linkage to covalently bind a pendant boronic acid-containing moiety to the HA monomer. Alternatively, the reaction with of the carboxylic acid group of a HA monomeric unit with a suitably reactive leaving group (e.g. a halogen atom such as chlorine) present on the boronic acid-containing moiety can also result in the formation of an ester linkage.

In an embodiment, the boronic acid-containing moiety is bound to the carboxy group of a HA monomer by an ester or amide linkage. In a further embodiment, the boronic acid-containing moiety is bound to the carboxy group of a HA monomer by an amide linkage.

Any suitable pendant boronic acid-containing moieties may be used in the polymer-drug conjugates of the present invention.

In an embodiment, the pendant boronic acid-containing moiety has the general structural formula I shown below:

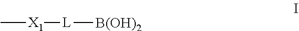

wherein L is linking group and X₁ is a functional group (e.g. —NH— or —O—) that connects the linking group L to the —C(═O)— of the carboxylic acid group present in the hyaluronic acid monomeric unit.

L may be any suitable linking group, e.g. an optionally substituted (1-10C)alkylene or an optionally substituted arylene linker. In an embodiment, L is an optionally substituted arylene linker, e.g. an optionally substituted phenylene linker.

X₁ may be any suitable functional group capable of linking L to the —C(═O)— of a carboxylic acid group of a hyaluronic acid monomeric units. In an embodiment X₁ is —O— or —NR— where R is H or (1-6C)alkyl. In another embodiment, X₁ is —O— or —NH—.

In an embodiment, the pendant boronic acid-containing moiety has the structural formula I shown above in which L is a phenylene linker, i.e. the pendant boronic acid-containing moiety has the structural formula Ia shown below:

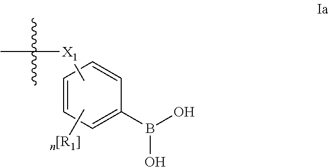

wherein

[symbol] denotes the point of attachment to the —C(═O)— group of the hyaluronic acid monomeric unit;

X₁ is a functional group (e.g. —NH— or —O—) that links to the C(O) atom of the carboxylic acid group present in the HA monomeric unit; and

R₁ is a substituent group selected from halo, cyano, nitro, hydroxy, mercapto, amino, carboxy, carbamoyl, (1-6C)alkyl, (2-6C)alkenyl, (2-6C)alkynyl, (1-6C)alkoxy, (2-6C)alkenyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulphamoyl, N,N-di-[(1-6C)alkyl]sulphamoyl, and wherein any alkyl moiety in any of the aforementioned groups is optionally substituted with one or more halo, cyano, nitro, hydroxy, mercapto, amino, carboxy, carbamoyl, (1-6C)alkoxy, (1-6C)alkylthio, (1-6C)alkylsulphinyl, (1-6C)alkylsulphonyl, (1-6C)alkylamino, or di-[(1-6C)alkyl]amino; and

n is 0, 1 or 2.

In an embodiment, R₁ is a substituent group that can coordinate to the boron atom, thereby increasing the stability of cyclic boronic esters (or related boron-containing linkers). In an embodiment, R₁ is a (1-6C)alkyl substituted by amino, (1-6C)alkylamino or di-[(1-6C)alkyl]amino. In another embodiment, R₁ is a (1-4C)alkyl substituted by amino.

In an embodiment, the pendant boronic acid-containing moiety has the structural formula Ib shown below

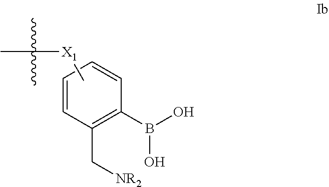

wherein
[symbol] denotes the point of attachment to the —C(═O)— group of the hyaluronic acid monomeric unit;
X₁ is a functional group (e.g. —NH— or —O—) that links to the C(O) atom of the carboxylic acid group present in the HA monomeric unit; and
each R group is selected from hydrogen or (1-4C)alkyl.

In a further embodiment, the pendant boronic acid-containing moiety has the formula Ic shown below:

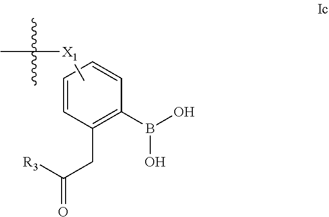

wherein
[symbol] denotes the point of attachment to the —C(═O)— group of the hyaluronic acid monomeric unit;
X₁ is —NH— or —O—; and
R₃ is a (1-4C)alkyl.

In a particular embodiment, the pendant boronic acid-containing moiety has the formula Id shown below:

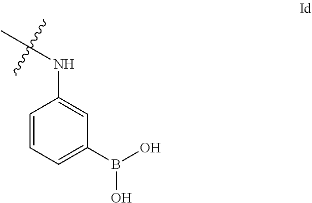 [SMILES: *Nc1cccc(B(O)O)c1]

wherein:
[symbol] denotes the point of attachment to the —C(═O)— group of the hyaluronic acid monomeric unit.

Suitably, 1 to 30% of the monomeric units present in the HA polymer backbone comprise pendant boronic acid-containing moieties. In an embodiment, 5 to 20% of the monomeric units present in the HA polymer backbone comprise pendant boronic acid-containing moieties. In a further embodiment, 8 to 15% of the monomeric units present in the HA polymer backbone comprise pendant boronic acid-containing moieties. In a further embodiment, 10 to 15% of the monomeric units present in the HA polymer backbone comprise pendant boronic acid-containing moieties.

Suitably, about 10% of the monomeric units present in the HA polymer backbone comprise pendant boronic acid-containing moieties.

### The Anticancer Drug

The anticancer drug may be any suitable anticancer drug known in the art that can either be:
(i) coupled directly to the boronic acid group of the modified HA polymers to form a boron-containing linkage as defined herein, or
(ii) coupled via a suitable linker group that can be coupled to the boronic acid group of the modified HA polymers to form a boron-containing linkage as defined herein.

Boronic acids are well known to react with 1,2-diols to form cyclic boronic esters or with 1,2-diamines boronic amides or vicinal aminoalcohols to form structurally related cyclic boronic amides. They can also react with 1,3-diketones through a similar mechanism.

The structures of an exemplary boronic ester and boronic amide are shown below.

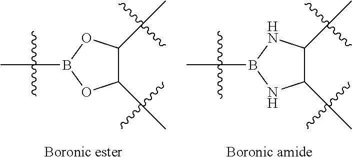 [SMILES: *B1OC(*)C(*)O1 ; *B1NC(*)C(*)N1]

Drugs that comprise catechol moieties (1,2-dihydroxybenzene moieties) or are linked to linker groups that comprise such moieties are particularly suited to coupling to the modified HA polymers of the present invention.

Examples of drug molecules that comprise such moieties include natural compounds such as quercetin, tannic acid, piceatannol, taxifolin, catechin and many other flavonoids. Curcumin and many of its derivatives can be bound through the central 1,3-diketone.

It will be appreciated that drug molecules that do not comprise suitable boronic binding groups, such as, for example, a diol (e.g. catechol) moiety or a diamine moiety, can still be coupled to the HA polymer via a linker group that comprises a suitable boronic binding moiety.

The linker group comprises functionality capable of binding to the drug molecule, and 1,2-diol (e.g. catechol), 1,2-diamine or vicinal aminoalcohol moieties capable of coupling to the boron-containing modified HA polymers of the present invention. Examples of suitable linker groups include dopamine and sugar diols, such as glucose, mannose and ascorbic acid.

An example of an anticancer drug molecule without suitable boronic binding groups is tirapazamine, which can be linked to the HA polymer via a suitable linker group, for example dopamine.

The modified HA polymers of the present invention may also be useful for the release of other bound molecules in environments characterised by an acidic pH, wherein the molecules comprise moieties suited to coupling to the modified HA polymers. Suitable such molecules may include aromatic diols such as pyrocatechol, pyrogallol, dopamine, epinephrine, norepinephrine and 2-hydroxy estradiol, or sugar diols such as glucose, mannose and ascorbic acid.

### The Polymer-Drug Conjugates

In the polymer-drug conjugates of the present invention, the anticancer drug is linked to the hyaluronic acid by a pH labile boronic ester or amide linkage.

Suitably, such linkages are formed by reacting a boronic acid group with a 1,2-diol or 1,2-diamine to form a boronic ester or amide linkage as previously described herein.

In an embodiment, the drug-polymer conjugate has the formula II shown below:

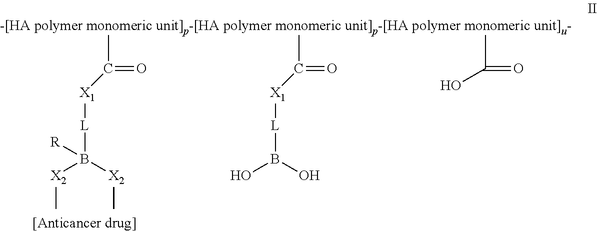

wherein

X₁ and L each have any one of the definitions set hereinbefore;

X₂ is O or NH;

R is as defined above;

p represents the proportion of HA monomeric units that are coupled to an anticancer drug via a boron-containing linkage (e.g. a boronic ester or amide linkage);

q represents the proportion HA monomeric units with uncoupled pendant boronic acid-containing moieties; and

u represents the proportion of HA monomeric units that do not comprise pendant boronic acid-containing moiety.

In an embodiment, X₁ is —O— or —NH—. In another embodiment, X₁ is —NH—.

In an embodiment, Lisa group of the formula:

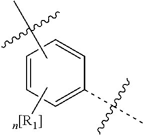

wherein
the solid bond denotes the point of attachment to X and dashed bond denotes the point of attachment to the boron atom; and
R₁ and n are as defined herein before.

In an embodiment, L is

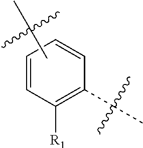

wherein
the solid bond denotes the point of attachment to X and dashed bond denotes the point of attachment to the boron atom; and
R₁ is as defined herein before.

In an embodiment, R₁ is selected from —CH₂—C(O)R₃ or —CH₂—NH₂.

In an embodiment, L is

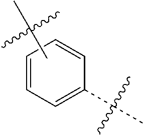

wherein
the solid bond denotes the point of attachment to X and dashed bond denotes the point of attachment to the boron atom.

In an embodiment, L is

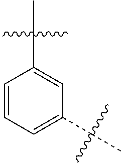

wherein
the solid bond denotes the point of attachment to X and dashed bond denotes the point of attachment to the boron atom.

In an embodiment, p is 0.5 to 20. In another embodiment, p is 1 to 10. In another embodiment, p is 3 to 7. In another embodiment, p is 4 to 6. In another embodiment, p is 5.

In an embodiment, q is 0.5 to 20. In another embodiment, q is 1 to 10. In another embodiment, q is 3 to 7. In another embodiment, q is 4 to 6. In another embodiment, q is 5.

In an embodiment, u is 60 to 99. In another embodiment, u is 70 to 99. In another embodiment, u is 80 to 99. In another embodiment, u is 85 to 95. In another embodiment, u is 90.

In an embodiment, p+q is from 1 to 40. In another embodiment, p+q is from 1 to 30. In another embodiment, p+q is from 1 to 20. In another embodiment, p+q is from 5 to 15.

In an embodiment, the drug-polymer conjugate has the formula II shown above where p, q and u are in a proportion of 5:5:90.

Suitably, X₁ is —NH—, X₂ is —O— and L is a linking group such as phenyl.

In an embodiment, the anticancer drug is a drug comprising a catechol moiety (e.g. quercetin and piceatannol) that reacts to form the boronic ester linkage, or a drug molecule that is linked to a linker group that comprises a catechol moiety.

As previously indicated, the cyclic boronic ester and amide linkages are pH labile such that the drug is stable at standard physiological pH but can be readily cleaved under mild acidic conditions, which is encountered in the endosome and/or interstitial space between tumour cells in solid tumours, as well as in hypoxic tumour environments.

In an embodiment, the anticancer drug is a drug with an enhanced anticancer effect under hypoxic conditions (e.g. tirapazamine).

A suitable pharmaceutically acceptable salt of a polymer-drug conjugate of the invention is, for example, an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

It is also to be understood that certain polymer-drug conjugates of the invention may exist in solvated as well as unsolvated forms, such as, for example, hydrated forms. It is to be understood that the invention encompasses all such solvated forms.

### Synthesis

In the description of the synthetic methods described herein, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

It will be appreciated that, during the synthesis of the polymer-drug conjugates of the invention in the processes defined herein, or during the synthesis of certain starting materials, it may be desirable to protect certain substituent groups in order to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

The synthetic processes of the present invention involve the reaction of a boronic acid binding moiety present in the anticancer drug molecule, or a linker attached thereto, with a pendent boronic acid-containing moiety present on a modified HA polymer as defined herein to form a boronic ester or amide linkage.

Suitable techniques to form such linkages are known in the art and are described further in the accompanying examples.

The present invention also provides a polymer drug-conjugate as defined herein obtained by, obtainable by, or directly obtained by a synthetic procedure as defined herein.

### Pharmaceutical Compositions

According to a further aspect of the invention there is provided a pharmaceutical composition which comprises a polymer-drug conjugate as defined hereinbefore, or a pharmaceutically acceptable salt or solvate thereof, in association with a pharmaceutically acceptable diluent or carrier.

The compositions of the invention may be in a form suitable for parenteral administration (for example as a sterile aqueous or oily emulsion for intravenous, subcutaneous, intramuscular, intraperitoneal or intramuscular dosing).

The compositions of the invention may be obtained by conventional procedures using conventional pharmaceutical excipients well known in the art. Thus, compositions intended for oral use may contain, for example, one or more diluents, buffers and/or preservative agents.

An effective amount of a polymer-drug conjugate of the present invention for use in therapy of proliferative disease is an amount sufficient to treat the cancer or slow its progression.

The amount of active ingredient that is combined with one or more excipients to produce a single dosage form will necessarily vary depending upon the host treated and the particular route of administration. For example, a formulation intended for parenteral administration to humans may contain, for example, from 0.5 mg to 5 g of active agent.

The size of the dose for therapeutic or prophylactic purposes of a polymer-drug conjugate of the present invention will naturally vary according to the nature and severity of the condition to be treated, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, optionally given, if required, in divided doses. In general lower doses will be administered when a parenteral route is employed. Thus, for example, for intravenous or intraperitoneal administration, a dose in the range, for example, 0.1 mg/kg to 30 mg/kg body weight will generally be used.

### Therapeutic Uses and Applications

The polymer-drug conjugates of the present invention may be used for the treatment of cancer. They are expected to be particularly suitable for the treatment of solid tumours. In addition, they are expected to be particularly suitable for the treatment of tumours in which CD44 is overexpressed, which are often those with the poorest prognosis, propensity to metastasis and higher resistance to chemotherapies (e.g. ovarian tumours (Biomolecules, 5 (2015) 3051); renal cell carcinomas (Scientific Reports 5 (2015) 13157), breast carcinomas (Int J Clin Exp Pathol 8 (2015) 11287); non-small cell lung cancer (Int J Clin Exp Pathol 7 (2014) 3632).

The type of cancer that can be treated will depend on the nature, potency and mechanism of action of the anticancer drug that is bound to the modified HA polymer.

Thus, the present invention provides a polymer-drug conjugate as defined herein, or a pharmaceutically acceptable salt or solvate thereof, or a pharmaceutical composition as defined herein, for use in the treatment of cancer. In a particular embodiment, the cancer is a human cancer.

In another aspect, the present invention provides a polymer-drug conjugate as defined herein, or a pharmaceutically acceptable salt or solvate thereof, or a pharmaceutical composition as defined herein, for use in the treatment of solid tumours.

In another aspect, the present invention provides the use of a polymer-drug conjugate as defined herein, or a pharmaceutically acceptable salt or solvate thereof, in the manufacture of a medicament for use in treatment of cancer. Suitably, the medicament is for use in the treatment of human cancers.

In another aspect, the present invention provides the use of a polymer-drug conjugate as defined herein, or a pharmaceutically acceptable salt or solvate thereof, in the manufacture of a medicament for use in the treatment of a solid tumour.

In another aspect, the present invention provides a method of inhibiting cell proliferation in vitro or in vivo, said method comprising contacting a cell with an effective amount of a polymer-drug conjugate as defined herein, or a pharmaceutically acceptable salt or solvate thereof.

In another aspect, the present invention provides a method of treating cancer in a patient in need of such treatment, said method comprising administering to said patient a therapeutically effective amount of a polymer-drug conjugate as defined herein, or a pharmaceutically acceptable salt or solvate thereof, or a pharmaceutical composition as defined herein.

In another aspect, the present invention provides a method of treating a solid tumour in a patient in need of such treatment, said method comprising administering to said patient a therapeutically effective amount of a polymer-drug conjugate as defined herein, or a pharmaceutically acceptable salt or solvate thereof, or a pharmaceutical composition as defined herein.

### Routes of Administration

The compounds of the invention or pharmaceutical composition comprising the active compound may be administered to a subject by any convenient route of administration, whether systemically/peripherally or topically (i.e. at the site of desired action).

### Combination Therapies

The anticancer treatment defined hereinbefore may be applied as a sole therapy or may involve, in addition to the polymer-drug conjugate of the invention, conventional surgery, radiotherapy or therapy with a further chemotherapeutic agent or a molecularly targeted agent. Such additional therapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) anti-invasion agents [for example c-Src kinase family inhibitors like 4-(6-chloro-2,3-methylenedioxyanilno)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl) piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];

(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) an endothelin receptor antagonist, for example zibotentan (ZD4054) or atrasentan; (viii) HSP90 inhibitors (for example, geldanamycin, radicicol or 17-N-Allylamino-17-demethoxygeldanamycin (17AAG));

(ix) antisense therapies, for example those which are directed to the targets listed above, such as ISIS 2503, an anti-ras antisense;

(x) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(xi) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the polymer-drug conjugates of this invention within the dosage range described hereinbefore and the other pharmaceutically-active agent within its approved dosage range.

According to this aspect of the invention there is provided a combination suitable for use in the treatment of a cancer (for example a cancer involving a solid tumour) comprising a polymer-drug conjugate of the invention as defined hereinbefore, or a pharmaceutically acceptable salt or solvate thereof, and another anti-tumour agent.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

### DESCRIPTION OF THE FIGURES

The following figures are referred to in the example section below:

FIG. 1. Left: ¹H NMR spectra of unmodified hyaluronic acid (below) and HAB (above); the inset presents a magnified version of the aromatic area of the latter spectrum, while the grey bar covers the water signal. Right: structure of HAB where numbers correspond to the location of the protons identified in the NMR spectrum. In a typical example, the ratio numerical ratio between functionalized and non-functionalized units n/m=0.316 (24:76).

FIG. 2. Dependency of FRET efficiency on pH for the FA-HAB/rhodamine-dopamine (FRET acceptor) complex. Fluorescence signal studied at λₑₓ=480 nm and λₑₘ=560 nm.

FIG. 3. Viability of LNCaP prostate cancer cells after 24, 48 and 72 hours of exposure at 37° C. to different concentrations of A: non-functionalized hyaluronic acid (HA) with average molecular weight of 200 kDa; B: HA derivatized with boronic groups (24% of the HA carboxylic residues, HAB); C: HAB further derivatized with quercetin to obtain the final macromolecular prodrug (0.396 mmol quercetin per gram of material, HABQ). D: quercetin (black symbols), compared to the HABQ (empty symbols). Please note that in the last panel the horizontal axis reports the molar concentration of the drug, which is in a free or bound form; as a result of the conjugation to the HABQ backbone the IC50 increases by at least one order of magnitude.

FIG. 4. A: Uptake of FA-HABQ in LNCaP cell line as a function of time. The 100% line corresponds to fluorescence emission of a 0.3 mg/mL solution of FA-HABQ. The plateau reached at 8 h and maintained at least until 24 h is typical of the CD44-mediated internalization of HA derivatives. B to E: Confocal images of LNCaP cell line after 0.5 (A), 2 (B), 4 (C) and 24 h (D) of contact with 0.3 mg/mL FA-HABQ; the green punctuated fluorescence corresponds to uptaken particles, which clearly move from a peripheral to a central (perinuclear) localization during the incubation.

FIG. 5. Confocal images of LNCaP cell line after 2 h of contact with FA-HABQ without (A) and with (B) pre-administration of anti-human CD44v6 antibody. The absence of any green fluorescence in B demonstrates the CD44-mediated nature of the HABQ internalization.

FIGS. 6. A, C and E: % wt. of the injected dose in various organs of prostate cancer bearing mice after 24 h of systemic administration of fluorescent prodrugs (A: FA-HABQ; B: FA-HABP) or hyaluronic acid carrier structure (C: FA-HAB). Please note that about 30% of the dose is still supposed to be circulating. B, D and F: Corresponding concentrations of FA-HABQ (B), FA-HABP (D) and FA-HAB (F) in the organs. Please note that e.g. the HABQ concentration in the tumor would roughly correspond to a 20-40 nM concentration of quercetin.

FIG. 7. A: Tumor mass in human prostate cancer-bearing mice as a function of time and of quercetin dosage. The arrows allow the comparison of formulations containing the same overall concentrations of quercetin, in order to visually compare the efficacy of the prodrug approach. B: The tumor volumes recorded with the use of free quercetin were divided by those obtained with the use of HABQ at the same overall quercetin concentration; the resulting differential tumoral volume is a quantitative indication of the efficacy of the prodrug approach, with higher numerical values implying a higher efficacy.

FIG. 8. Screening of human cancer cell lines in terms of production of cytochrome P450 reductase (Western blots); toxicity of HAB-TPZ-DOPA to various cancer cell lines after 3 h exposure to the conjugate as a function of the molar concentration of TPZ groups, expressed as the corresponding IC50 values.

FIG. 9. 72 h viability of eight cancer cell lines after 3 h exposure to TPZ-DOPA under both normoxic (air) and hypoxic (0.1% oxygen) conditions, expressed as percentage cell survival compared to control.

FIG. 10. 72 h viability of eight cancer cell lines after 3 h exposure to HAB-TPZ-DOPA under both normoxic (air) and hypoxic (0.1% oxygen) conditions, expressed as percentage cell survival compared to control.

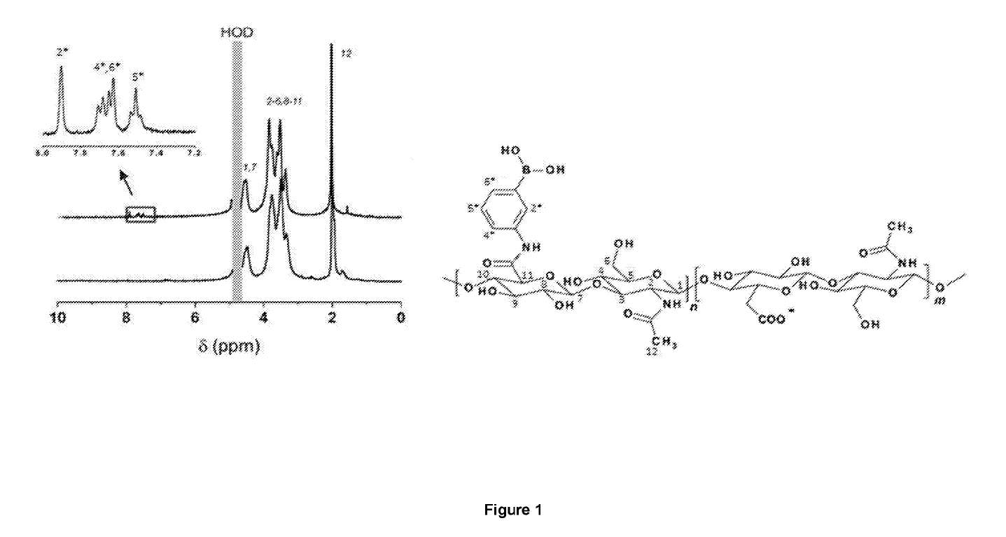

### EXAMPLES

In the following examples, quercetin has been employed as a model drug that a) contains a catechol group, b) has known chemotherapeutic activity, c) has a poor therapeutic performance due to its insolubility and also to nephrotoxicity; to date these issues have been only partially overcome e.g. with liposomal formulations¹. Piceatannol has also been used in example 5 (accumulation in solid tumours) and curcumin in example 8. Tirapazamine has been used as an example of a hypoxia-activated anticancer drug that can be conjugated to HAB (example 7).

### Example 1—Preparation of the Macromolecular Prodrug of Quercetin

### Summary

The following procedures illustrate the preparation of a macromolecular prodrug based on hyaluronic acid with 200 kDa average molecular weight, and a quercetin loading of 0.396 mol/g of material. Other molecular weights, quercetin loadings, or quercetin/boronate ratios can be easily prepared by varying the preparative conditions in obvious ways.

Methods

Non-Fluorescently Labeled Boronated HA (HAB)

40 mg (100 μmol of carboxylic groups) of hyaluronic acid (viscosimetric average molecular weight: 200 kDa) were dissolved in 10 mL of distilled water. 28 mg of 4-(4,6-Dimethoxy-1,3,5-triazin-2-yl)-4-methylmorpholinium chloride (DMTMM, 100 μmol) was added to the solution. After 10 minutes, 1 mL of a 8.6 mg/mL solution of 3-aminophenyl boronic acid (3-APBA, 49.6 μmol) in water was added to this mixture and allowed to stir overnight at room temperature. The product was precipitated in cold ethanol and then dialysed against distilled water using regenerated cellulose (RC) Spectra/Por Dialysis membrane tubing (MWCO=10,000 g/mol), in order to remove excess of DMTMM, byproducts and unreacted 3-APBA. The material was finally freeze dried after completion of dialysis, which was recognized from the drop in conductivity in the dialysis water. Average yield (weight of recovered material/weight of recoverable material)=75-85%. The degree of derivatization was obtained from NMR spectra, by integrating the area below the peaks for the 3-APBA and the acetyl peak for HA and comparing these integrated values using the acetyl group of HA as a reference peak and the aromatic signals of APBA as sample peak, according to the following equation:

$$\frac{\begin{matrix}Integral of \\ reference peak\end{matrix}}{\begin{matrix}Number of protons \\ of reference peak\end{matrix}} = \frac{\begin{matrix}Integral of sample peak / \\ Number of protons of sample peak\end{matrix}}{Molar fraction of modified units}$$

24% of the carboxylic groups were modified with boronic acids (¹H-NMR analysis), corresponding to 0.56 mmol of boronates per gram of material.

¹H-NMR (D₂O): δ=7.76-7.81 (H 2*), 7.49-7.6 (H 4* and 6*), 7.36-7.42 (H 5*), 4.3-4.5 (H 1 and 7), 3.1-3.8 (H 2 to 6 and 8 to 11), 1.8-1.9 ppm (H 12).

Fluorescently Labeled Boronated HA (FA-HAB)

50 mg of HABQ were dissolved in 40 ml of distilled water and diluted with 20 mL of DMSO. A solution of 25 mg (0.075 mmol) of fluoresceinamine, 25 μL of acetaldehyde (0.9 μmol) and 25 μL of cyclohexyl isocyanide (0.2 μmol) in 100 μL of DMSO was added to the HABQ solution. After stirring overnight, the polymer was precipitated three times in cold ethanol (disappearance of fluorescein fluorescence) and freeze dried. Yield: 80-85% in weight. The degree of labeling was determined fluorimetrically by using filters at 485±20 nm (excitation) and 528±20 nm (emission) and a calibration with free fluoresceinamine and 0.29% of the total carboxylic groups resulted functionalized with pendant fluorophores.

Quercetin Macromolecular Prodrug (HABQ)

5 mL of a solution 4 mg/mL of HAB (total of 11 μmol of boronate groups) in 100 mM phosphate buffer at pH 8 were mixed to 5 mL of a 2.1 mg/mL quercetin solution in phosphate buffer at pH 8 containing 10% v/v of DMSO (34 μmol of quercetin, corresponding to quercetin/boronate molar ratio 3:1). The solution was stirred for 30 min and then precipitated in cold ethanol, to remove unreacted quercetin. After evaporation of ethanol HABQ was dissolved in 5 mL of distilled water and freeze dried. The amount of drug loaded on polymer was measured first by releasing quercetin from the polymer structure (1 mg of polymer was dissolved in 1 mL of 10 mM acetate buffer at pH 4), and then determining the amount of free quercetin via HPLC (HPLC Agilent 1200 Infinity series equipped with a C18 column (2.1 mm×250 mm) and a UV detector working at 370 nm, as reported in literature²) with the help of a calibration curve. Quercetin load: 0.396 mmol quercetin per gram of material (70% functionalization of boronic acid residues).

The fluorescently labeled macromolecular prodrug (FA-HABQ) was prepared identically, replacing HAB with FA-HAB.

Piceatannol Macromolecular Prodrug (HABP)

5 mL of a solution 4 mg/mL of HAB (total of 11 μmol of boronate groups) in 100 mM phosphate buffer at pH 8.6 were mixed to 5 mL of a 1.6 mg/mL piceatannol solution in phosphate buffer at pH 8.6 containing 10% v/v of DMSO (33 μmol of piceatannol, corresponding to piceatannol/boronate molar ratio 3:1). The solution was stirred for 30 min and then precipitated in cold ethanol, to remove unreacted piceatannol. After evaporation of ethanol HABP was dissolved in 5 mL of distilled water and freeze dried. The amount of drug loaded on polymer was measured first by releasing piceatannol from the polymer structure (1 mg of polymer was dissolved in 1 mL of 10 mM acetate buffer at pH 4), and then determining the amount of free piceatannol via HPLC (HPLC Agilent 1200 Infinity series equipped with a C18 column (2.1 mm×250 mm) and a UV detector working at 325 nm, as reported in literature²) with the help of a calibration curve. Piceatannol load: 0.438 mmol piceatannol per gram of material (77% functionalization of boronic acid residues).

The fluorescently labeled macromolecular prodrug (FA-HABP) was prepared identically, replacing HAB with FA-HAB.

### Example 2—Proof of Principle that the New Chemical Entity can Release a Catechol-Based Compound at Acidic pH

### Summary

The following example shows that an acidic pH can induce the release of catechols in a free form from its complex with a boronate-containing hyaluronic acid (HAB).

Introducing a Fluorescence Resonance Energy Transfer (FRET) donor (fluoresceine) on a boronic acid-containing macromolecule and a FRET acceptor (rhodamine) on a catechol, we have used FRET efficiency as a measure of the spatial proximity of the two groups participating to the formation of the catechol/boronate complex in the macromolecular model compound: if FRET occurs, the rhodamine emission at 560 nm will be observed upon excitation of fluorescein.

Methods

Preparation of a Catechol-Bearing Fluorophore as a FRET Acceptor

Dopamine hydrochloride was covalently conjugated to the fluorophore rhodamine isothiocyanate in non-oxidizing conditions, following a literature procedure³; the resulting construct was referred to as dopamine-rhodamine conjugate. First methanol and other required solutions were degassed to protect the oxidation dopamine. Dopamine hydrochloride (37.9 mg/20 μmol) was dissolved in 10 mL of degassed methanol, adding an excess of triethylamine (60 μL/40 μmol) to convert it to the free base form and stirring the solution for 30 min under argon. Rhodamine isothiocyanate (106 mg/20 μmol) was then added to the solution and the mixture was left under stirring at room temperature for 4 hours.

Preparation the Macromolecular Model Compound

20 mg of FA-HAB (corresponding to 0.024 mmol of boronate groups) were dissolved in 25 mL of degassed 10 mM phosphate buffer at pH 8; 10 mL of a 0.7 mg/mL solution of the FRET acceptor (corresponding to 0.040 mmol of catechols) were added drop-wise for 30 min. The reaction mixture was then purified by dialysis against distilled water (5 kDa cut-off; Spectrum Laboratories, Inc.) while bubbling argon for seven days (no rhodamine fluorescence in the dialysis medium); the solution was finally freeze dried to yield 15 mg of product.

FRET Experiments

Protocol

The macromolecular model compound was dissolved at a concentration of 100 μg/mL.in 10 mM acetic acid buffer at pH 3.7 and 5, in 10 mM Phosphate buffer at pH 6, 7 and 7.5, in Tris buffer at pH 8 and 8.5. FRET measurements were conducted in a 96-well black plate, using an excitation 480 nm±10, (fluorescein excitation peak) and measuring the emission intensity at 560 nm±10 (rhodamine emission peak). The measurements were conducted at 25° C. in a Tecan Infinite M200 plate reader, equipped with I control software).

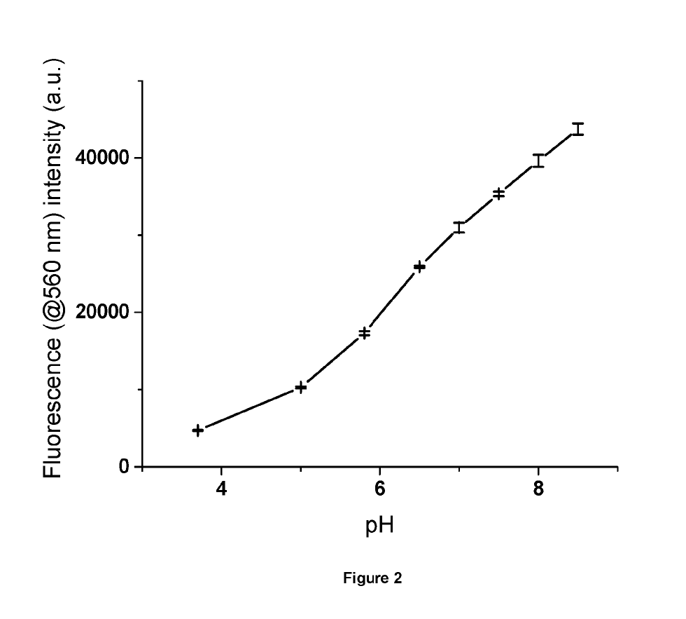

Discussion (See FIG. 2)

The FRET effect strongly reduced with decreasing pH, demonstrating the acid pH-induced release of the catechol-containing molecule.

### Example 3—Proof of Principle of the Safety of the New Chemical Entity

### Summary

The following examples show that the carrier structure (HAB) has very low cytotoxicity and also the quercetin-containing macromolecular prodrug (HABQ) has a low toxicity, if quercetin is maintained in a bound form, i.e. if the construct is kept at neutral pH.

The macromolecular prodrug used for these experiments was based on hyaluronic acid with viscosimetric average molecular weight of 200 kDa bearing 0.565 mmol of boronic acid units per gram of material and loaded with 0.396 mmol quercetin per gram of material (1:0.7 boronic acid/quercetin molar ratio).

### Methods

The cytotoxicity of the HABQ was evaluated on prostate cancer cells (LNCaP cell line) evaluating their mitochondrial dehydrogenase activity by the means of a modified MTT [3-(4,5-dimethyldiazol-2-yl)-2,5-diphenyltetrazolium bromide] method according to the manufacturer's instructions (Dojindo Molecular Technologies Inc., Rockville, Md.). Prostate cancer cells were seeded in 96-well plates at a density of 10,000 cells per well in RPMI-1640 medium (Gibco) containing 10% FBS, 1% Pen-Strep and 2 mM L-Glutamine at 37° C. in a humidified 5% CO₂ atmosphere. The cytotoxicity was evaluated using the modified MTT assay at 24, 48 and 72 h as a function of the macromolecular prodrug concentration. At the end of the incubation period the cells were washed three times with PBS at pH 7.4 and incubated with 100 μl of a MTT solution (0.5 mg/ml in cell culture medium) for 4 h at 37° C. The absorbance readings were acquired at a wavelength of 450 nm with the Tecan Infinite M200 plate-reader using I-control software. The relative cell viability (%) was calculated by the formula [A]ₜₑₛₜ/[A]_{control}×100, where “[A]ₜₑₛₜ” is the absorbance of the test sample, and “[A]_{control}” is the absorbance of the control cells incubated solely with culture medium. After evaluating cell cytotoxicity, the total protein content was measured by using the Micro BCA protein assay kit (Pierce). Briefly, the cells were washed with ice-cold PBS, and incubated for 15 min in 150 μL cell lysis buffer (0.5% v/v Triton X-100 in PBS), to which 150 μL of Micro BCA protein assay kit reagent (prepared following the instructions of the manufacturer) were added. The absorbance at 562 nm was finally measured on a plate reader. The cytotoxicity measurements were then normalized by the amount of total protein content in each well.

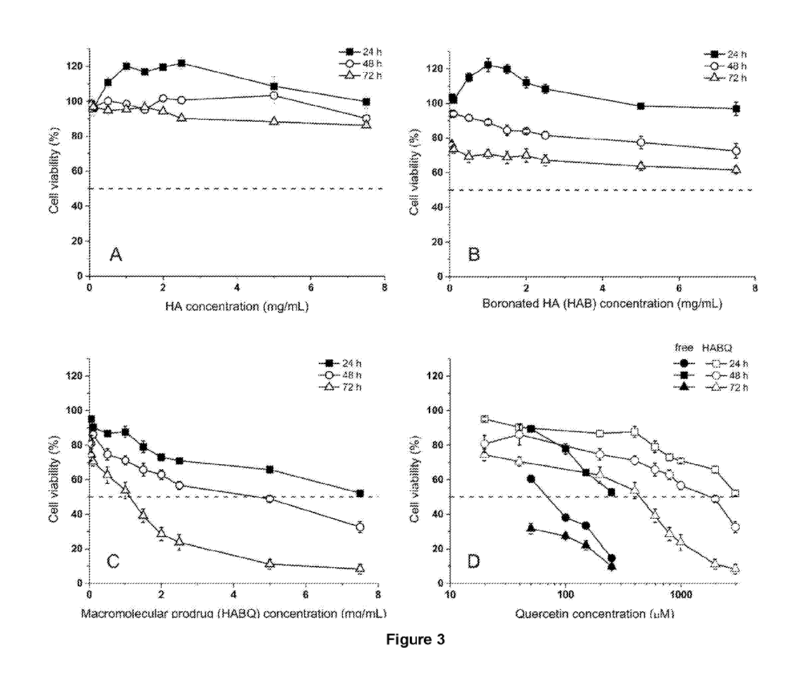

### Discussion (See FIG. 3)

A) The pH of the cell culture was measured after 24, 48 and 72 h of incubation of cancer cells with the HABQ with concentrations up to 7.5 mg/mL or in its absence; independently on its presence, a pH value of 7.8 was recorded after 24 and 48 h, and of 7.5 after 72 h of contact with a 7.5 mg/mL concentration of prodrug. Therefore we can assume that in the experiments performed with HABQ, quercetin is essentially present only as macromolecular prodrug (and not as a free drug due to the non-acidic pH).

B) The HA carrier structure (HAB) showed negligible toxicity up to high concentration (IC50>>8 mg/mL even after 72 h incubation), which means that the carrier structure itself can be seen as biologically benign.

C) The macromolecular prodrug HABQ showed low toxicity, with IC50≈5-7.5 mg/mL for incubation times up to 48 h, whereas the viability decreased considerably at 72 h (IC50=1-1.5 mg/mL). The IC50 values correspond to a total quercetin concentration between 0.5 mM (72 h) and >2 mM (24 h).

Free quercetin, on the other hand, has a much higher toxicity with IC50 values typically 1 to 3 orders of magnitude lower than those recorded for HABQ: 5 μM in NCI-H209 lung cancer cells (24 h)⁴, 10 μM in MCF7 breast cancer cells (72 h)⁵, 20 μM in 16-F10 melanoma cells (72 h)⁶, 150-200 μM in HK1 and C66-1 squamous nasopharynx carcinoma cells (72 h)⁷. Therefore we conclude that the low toxicity of HABQ is due to the benign character of the macromolecular prodrug form.

### Example 4—Proof of Principle that the New Chemical Entity Targets CD44 in Tumoral Cell Lines

### Summary

The following procedures show that the quercetin-containing macromolecular prodrug (HABQ) is uptaken by tumoral cells and exploit the same receptor (CD44)-mediated internalization mechanism as hyaluronic acid.

This study employed the same FA-HABQ derivative described at point B.

### Uptake Quantification

### Methods

LNCaP cells were cultured in RPMI-1640 medium (Gibco) containing 10% FBS, 1% Pen-Strep, L-Glutamine 2 mM at 37° C. in a humidified 5% CO₂ atmosphere. For uptake experiments, 5×10³ cells/well were seeded in 24-well plate and allowed to grow for 24 h. The medium was then replaced with 0.1 mL of a 0.3 mg/mL solution of FA-HABQ in culture medium and allowed to incubate for a time comprised between 0.5 and 24 h. Cells were then washed twice with PBS (pH 7.4) and after specified time intervals, the experiments were terminated by removing the supernatant, washing the cells three times with 10 mM PBS and lysing the cells with 0.1 mL of 0.5% Triton X-100 in 0.2 N NaOH. The membrane-bound and internalized FA-HABQ was quantified by analyzing the fluorescence of the cell lysate (λₑₓₑ=485 nm, λₑₘ=535 nm), employing a calibration with 0.001-0.6 mg/mL FA-HABQ dispersed in a cell lysate solution (10⁶ untreated cells dissolved in 1 mL of the Triton X-100/0.2 N NaOH solution).

### Discussion

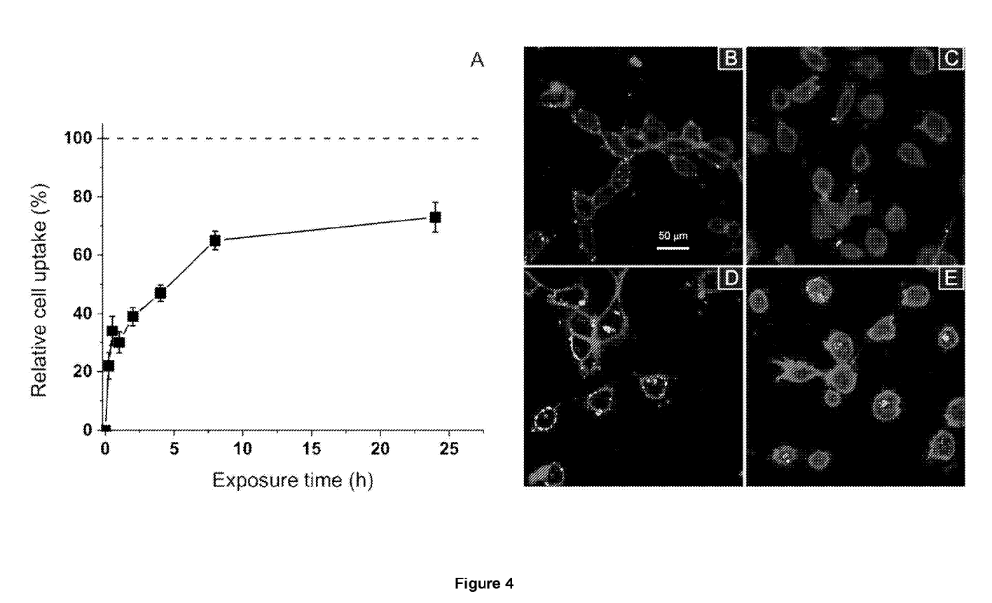

The uptake of FA-HABQ reached a plateau after approximately 8 h (FIG. 4A); this phenomenon of saturation is common in HA-containing materials and is due to the lack of recycling of HA receptors, in particular for CD44, leading to their disappearance from the cell surface for a period up to 24-36 hours. The data therefore suggest a receptor-mediated internalization in the cancer cells.

### Imaging and Competitive Inhibition

### Methods

LNCaP cells were cultured in RPMI-1640 medium (Gibco) containing 10% FBS, 1% Pen-Strep, L-Glutamine 2 mM at 37° C. in a humidified 5% CO₂ atmosphere. After, 5×10³ cells/well were seeded in a 24-well plate and allowed to grow for 24 h. The medium was then replaced with anti human CD44v6 antibody IgG1 (clone VFF7, Abcam, stock concentration 1 mg/mL) diluted 1/200 for 1 h. The wells were then washed two times with PBS and the medium was finally replaced with a 0.3 mg/ml FA-HABQ solution in culture medium and incubated for 2 h.

Subsequently, cells were thoroughly rinsed three times with PBS and fixed with a 2.5% glutaraldehyde in PBS for 20 min. Membrane staining was obtained by using Concanavalin A Tetramethylrhodamine Conjugate (Invitrogen, Life Technology) at a final concentration of 100 μg/mL. After washing in PBS, cells were blocked with 1% BSA in PBS for 20 min and washed three times with PBS. Using a Confocal Microscope (C1 Nikon) equipped with a EZ-C1 Software for data acquisition and 60× oil immersion objective, the macromolecular prodrug was imaged through excitation/emission at 492/518 nm for, and the cell membrane with excitation/emission at 555/580 nm.

### Discussion

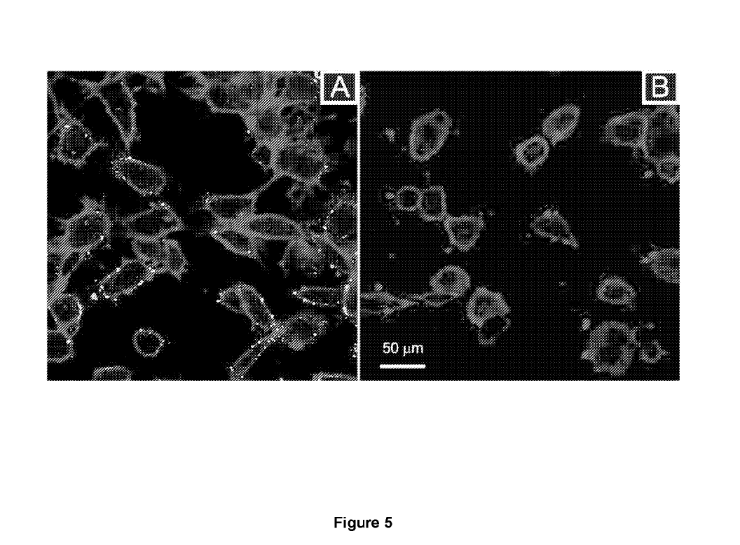

The fluorescence of FA-HABQ (green in FIG. 4B-E) was visibly associated to peripheral intracellular compartments at early time point (0.5-4 h, FIG. 4B-D) whereas a more central, probably lysosomal localization was recorded after 24 hours (FIG. 4E). It was finally shown (FIGS. 5A and B) that the pre-incubation with CD44v6 antibody completely blocked the uptake of the prodrug, therefore finally demonstrating HABQ to be uptaken in a CD44-dependent fashion.

### Example 5—Proof of Principle that the New Chemical Entity Accumulates in Solid Tumors

### Summary

The following example illustrates that quercetin- or piceatannol-containing macromolecular prodrugs (HABQ) have a preferential accumulation in human tumoural tissues ectopically implanted in an immunodeficient rodent model. This accumulation is considerably higher than that of the drug-free carrier structure (HAB).

Note: piceatannol is a catechol-containing compound with proven antileukaemic activity, but also the metabolite and probably the responsible of the activity of resveratrol, a well-known cancer preventative agent^{8,9}.

### Methods

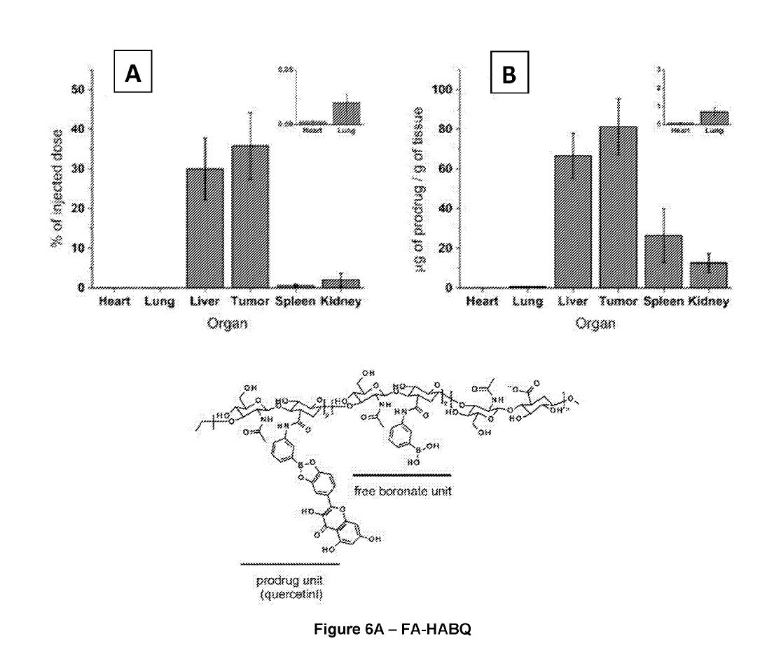

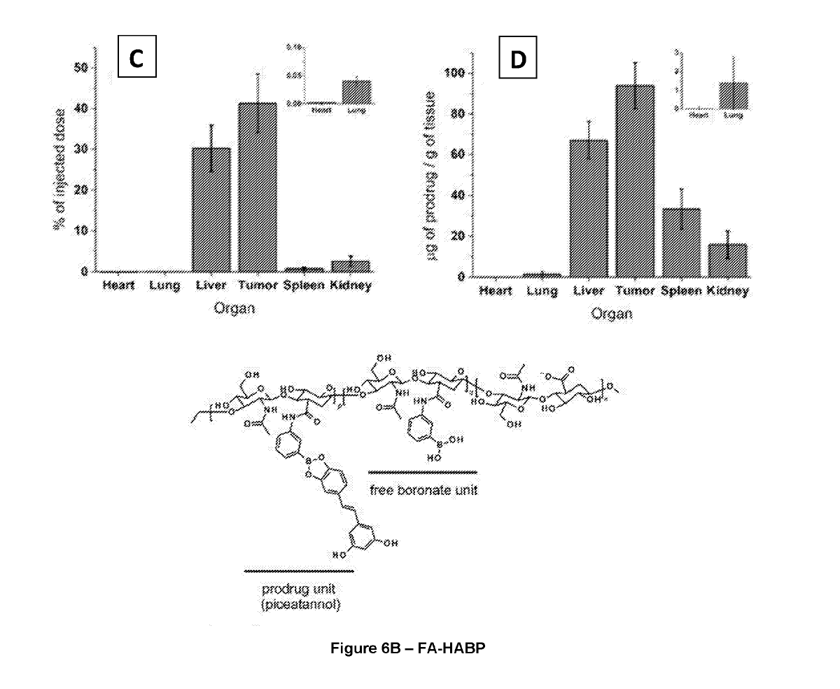

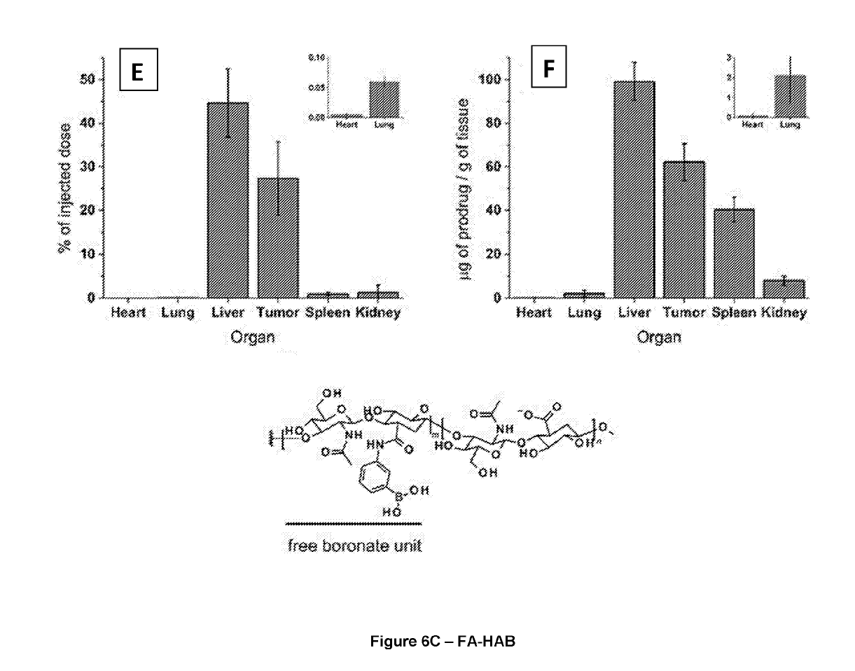

Tumor-bearing mice were prepared by injecting a suspension of LNCaP cells 1×10⁶ in 100 μL of saline physiological solution into the subcutaneous space of athymic nude mice dorsa (seven weeks old, 20-25 g). After 14 days of appropriate tumor growth, FA-HABQ (0.396 mmol of quercetin per gram of material, 1:0.7 boronic acid/quercetin molar ratio), FA-HABP (0.438 mmol of piceatannol per gram of material, 1:0.8 boronic acid/piceatannol molar ratio) and FA-HAB were injected into the tail vein of the tumor-bearing mice (n=10/group) at a dose of 10 mg/kg, which corresponds to 3.96 μmol of quercetin or 4.38 μmol of piceatannol/kg of weight. After 24 h the animals were sacrificed and tumor, spleen, heart, liver, lungs, kidneys were removed, weighed, lysed with sodium dodecyl sulfate (SDS) solution 5% w/v in water for 1 hour and processed for the analysis of fluorescence intensity. The values were expressed as μg of fluorescent macromolecular prodrug/g of tissue (FIG. 6, A) and percentage of injected dose per organ (% ID/organ) (FIG. 6, B) and were calculated on the basis of normalization of a standard curve with a range of concentration of FA-HABQ between 5 mg/mL up to 0.05 μg/mL.

### Discussion

Both the macromolecular prodrugs and the boronated HA carrier showed no significant accumulation in cardiac and pulmonary tissues, minor amounts could be identified in the kidneys and spleen, whereas the highest accumulation was recorded in the tumoral tissue and in liver.

First, there is a major pharmacokinetic difference between macromolecular prodrugs and free drugs: quercetin^{10,11} and piceatannol⁸ show a strong renal and pulmonary accumulation^{10,11}. Second, the distribution of macromolecular prodrugs appears depends on that of HA, which localizes in the liver with great preference than in any other organ¹²⁻¹⁴, and behaves similarly in solid tumors^{15,16}; however, the HA carrier alone showed a significantly higher accumulation in the liver, whereas the highest localization of the prodrugs was in the tumors.

### Example 6—Proof of Principle of the Anti-Tumoral Effects of the New Chemical Entity and of its Advantage Over Free Drug

The following example shows that the quercetin-containing macromolecular prodrug (HABQ) determines a significant reduction of the growth of ectopically implanted tumours in an immunodeficient rodent model; this reduction is considerably larger than that of free quercetin. This effect compounds with much lower toxicity of the delivery formulation.

### Methods

Tumor-bearing mice were prepared by injecting a suspension of LNCaP cells 1×10⁶ in saline physiological solution (100 μL) into the subcutaneous dorsa of athymic nude mice (seven weeks old, 20-25 g). After fourteen days of appropriate tumor growth, LNCaP bearing mice were divided into eight groups (n=10 for each group). Treatment was initiated when the tumor volume reached 90 mm³. The mice were then i.v. injected with 200 μL of the following solutions: HABQ (HA molecular weight: 200 kDa; loading of 0.396 mmol of quercetin per gram of material; 1:0.7 boronic acid/quercetin molar ratio) in PBS to obtain doses of 25 and 50 mg/kg (respectively corresponding to 3 and 6 mg/kg of quercetin), free quercetin in PBS containing 11% wt. DMSO to obtain doses of 3, 6, 25 mg/Kg, HAB to obtain a dose of 50 mg/Kg, and DMSO 11% wt. in PBS. Intravenous injections, administered in tail vein, were performed every 3 day for a total period of 21 days of treatment and then sacrificed according to ethical laws in force, following a literature method¹. The tumor size was determined by caliper measurement of the largest and perpendicular diameters during the time of treatment. Tumor volumes were calculated according to the formula: V=a×b²×0.52, where a is the largest superficial diameter and b is the smallest superficial diameter, according to a literature procedure¹⁷.

### Discussion

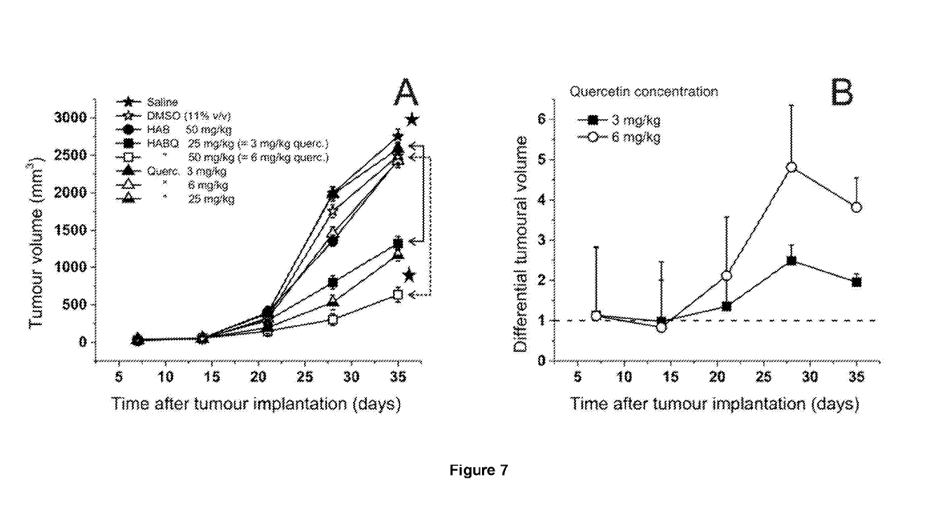

The decrease in tumor growth rate was particularly significant with both HABQ dosages employed, which corresponded to quercetin doses of 3 and 6 mg/kg, whereas comparable results could be obtained only with a dose of 25 mg/kg of free quercetin (FIG. 7A). In a comparison of the efficacy of the prodrug vs. that of the free drug, it was apparent that for the same quercetin dosage the prodrug approach produced a 2-(3 mg/kg dose) or 4-fold (6 mg/kg) better result.

### Example 7—Proof of Principle that the Conjugation of a Hypoxia-Activatable Group Preserves its Responsive Activity after the Formation of the Macromolecular Prodrug

### Summary

The following example shows that tirapazamine (TPZ), a drug with enhanced toxicity under hypoxic conditions, can be converted into a macromolecular prodrug via a catechol-containing linker group (dopamine, DOPA), and that the HAB-TPZ-DOPA conjugate possesses hypoxia-activated cytotoxicity.

### Methods

### Synthesis of Tri-TBDMS Protected Dopamine (100)

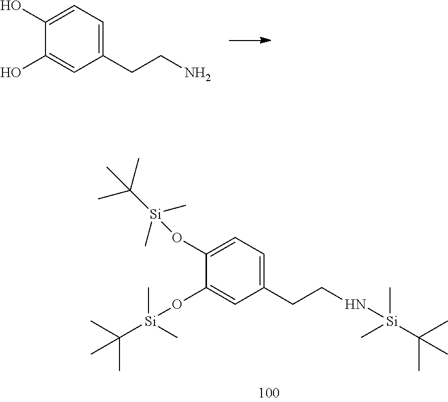 [SMILES: NCCc1ccc(O)c(O)c1 ; CC(C)(C)[Si](C)(C)NCCc1ccc(O[Si](C)(C)C(C)(C)C)c(O[Si](C)(C)C(C)(C)C)c1]

Dopamine hydrochloride (12.6 g, 66.4 mmol, 1.0 eq.) and triethylamine (46.3 mL, 335.0 mmol, 5.0 eq.) were dissolved in dry acetonitrile (131.3 mL) under an argon atmosphere. The solution was cooled to 0° C. in an ice bath, and then a solution of t-butyldimethylsilyl chloride (40.1 g, 266.1 mmol, 4.0 eq.) in dry acetonitrile (131.3 mL) was added dropwise. The reaction mixture was stirred at room temperature overnight. The solvent was then evaporated under vacuum, and the resulting crude product was partitioned between water (100 mL) and CH₂Cl₂ (200 mL). The organic layer was then separated, and the water phase extracted with CH₂Cl₂ (2×200 mL). Finally, the combined organic phases were washed with 1 M citric acid buffer solution (50 mL, pH 5), dried over NaSO₄, and concentrated on the rotary evaporator. The resulting crude product was dried overnight in a vacuum oven resulting in 31.11 g of tri-TBDMS protected dopamine 100 as a light brown wax. (Yield: 97%). ¹H NMR (400 MHz, CDCl₃) δ ppm: 0.09 (s, 6H), 0.19 (s, 12H), 0.92 (s, 9H), 0.98 (s, 18H), 1.76 (br. s, 1H), 2.62 (t, 2H), 2.90 (t, 2H), 6.63 (dd, 1H), 6.66 (d, 1H), 6.75 (d, 1H); IR (cm⁻¹) (solid): 2954, 2929, 2852, 1575, 1510, 1293, 1252, 907, 834, 777.

### Synthesis of Di-TBDMS Protected Dopamine (101)

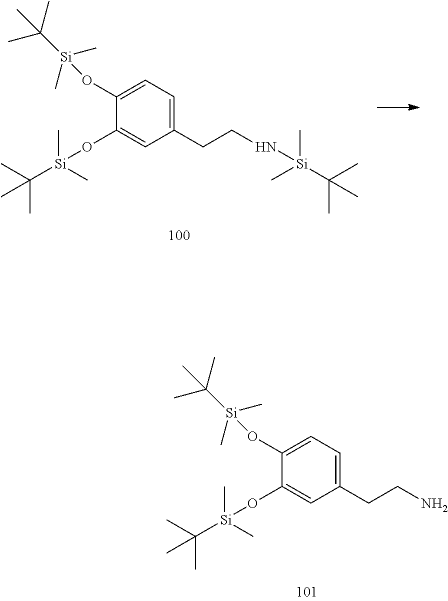

100 (7.7 g, 15.98 mmol, 1.0 eq.) was dissolved in 154 mL of 0.5 M HCl in isopropyl alcohol at 0° C. under an argon atmosphere. The reaction mixture was stirred for 36 hours, after which the small amount of solid precipitate formed was filtered off. Then, 8.16 g of finely ground Na₂CO₃ (2.0 eq. compared to HCl) were added and the solution was stirred overnight at 0° C. The solid was removed by filtration and the solvent concentrated on the rotary evaporator. The resulting crude product was dissolved in CH₂Cl₂, finely ground CaCl₂ (ca 1 g) was added to the solution and left to stir overnight at room temperature. Finally, after removal of the solid, the solution was concentrated on the rotary evaporator resulting in 5.43 g of di-O-TBDMS protected dopamine 101 as a slightly brown solid. (Yield: 89%). ¹H NMR (400 MHz, CDCl₃) δ ppm: 0.16-0.20 (m, 12H), 0.96-1.00 (m, 18H), 2.95-3.02 (m, 2H), 3.19 (m, 2H), 6.67 (s, 1H), 6.68-6.71 (m, 1H), 6.74-6.78 (m, 1H), 8.43 (br. s., 2H); IR (cm⁻¹) (solid): 2954, 2929, 2857, 1599, 1509, 1290, 1252, 902, 836, 770.

### Synthesis of Tirapazamine Active Ester (102)

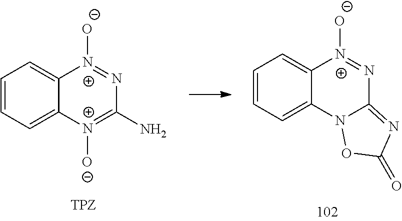 [SMILES: Nc1n[n+]([O-])c2ccccc2[n+]1[O-] ; O=c1nc2n[n+]([O-])c3ccccc3n2o1]

A suspension of finely ground tirapazamine (TPZ) (500 mg, 2.81 mmol, 1.00 eq.) in dry toluene (12.5 mL) was refluxed under an argon atmosphere. Triphosgene (865 mg, 2.91 mmol, 1.05 eq.) dissolved in dry toluene (37.5 mL) was added to the reaction flask in three equal portions at 30 minute intervals. After the final addition, the reaction mixture was stirred for 10 additional minutes, and then allowed to reach room temperature. Afterwards, hexane (80 mL) was added, and the yellow solid precipitate recovered and thoroughly washed with hexane (three times), before being dried under vacuum to produce 486 mg of tirapazamine active ester 102 as a yellow solid. (Yield: 85%). ¹H NMR (400 MHz, DMSO-d₆), δ ppm: 7.75 (ddd, 1H), 8.01 (dd, 1H), 8.21 (ddd, 1H), 8.43 (dd, 1H); IR (cm⁻¹) (solid): 3079, 1811, 1609, 1537, 1483, 1434, 1342.

### Synthesis of Di-TBDMS Protected TPZ-DOPA (103)

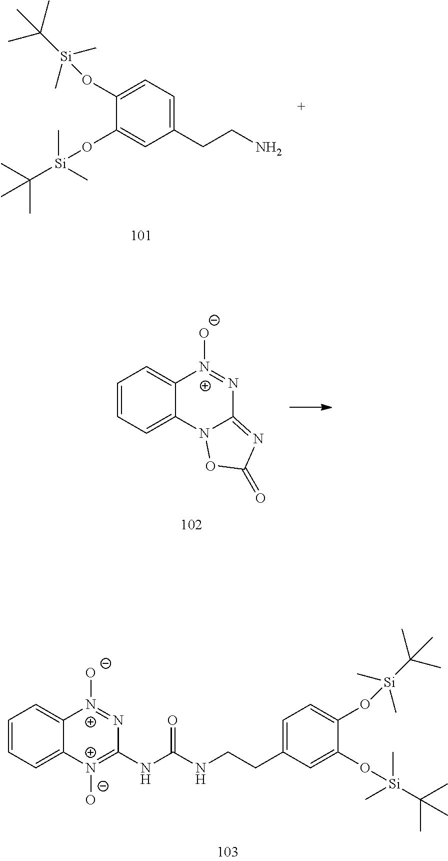 [SMILES: CC(C)(C)[Si](C)(C)Oc1ccc(CCN)cc1O[Si](C)(C)C(C)(C)C ; O=c1nc2n[n+]([O-])c3ccccc3n2o1 ; CC(C)(C)[Si](C)(C)Oc1ccc(CCNC(=O)Nc2n[n+]([O-])c3ccccc3[n+]2[O-])cc1O[Si](C)(C)C(C)(C)C]

Tirapazamine active ester 102 (465 mg, 2.27 mmol, 1.00 eq.) and di-TBDMS protected dopamine 101 (1000 mg, 2.62 mmol, 1.15 eq.) were dissolved in dry DMF (34 mL) under an argon atmosphere. The reaction mixture was then cooled to 0° C., and then triethylamine (548 μL, 3.91 mmol, 1.72 eq) was added dropwise. The reaction mixture was stirred at 0° C. for 30 minutes, and then allowed to reach room temperature and stirred overnight. Afterwards, the solvent was concentrated under vacuum and the resulting crude product was dissolved in 1.5% acetic acid in ethanol (60 mL), and the solvent concentrated under vacuum. The resulting solid was loaded onto silica gel (70-230 mesh) and purified by column chromatography (gradient: from 49.75:49.75:0.5 to 74.75:24.75:0.5 ethyl acetate:hexane:acetic acid). Traces of acetic acid in the final product were removed by azeotropic evaporation with toluene to give 1.03 g of di-TBDMS protected dopamine-TPZ 103 as an orange solid. (Yield: 77%). ¹H NMR (400 MHz, DMSO-d₆), δ ppm: 0.17 (s, 12H), 0.94 (s, 18H), 2.67 (t, 2H), 3.30-3.37 (m, 2H), 6.70-6.80 (m, 3H), 7.63 (t, 1H), 7.76 (t, 1H), 8.04 (t, 1H), 8.26 (d, 2H), 8.30 (d, 2H), 9.9 (br. s., 1H); IR (cm⁻¹) (solid): 3358, 3079, 2930, 2857, 1705, 1536, 1508, 1485, 1413, 1332, 1290, 1253, 1161, 1090, 906, 835, 777.

### Synthesis of TPZ-DOPA (104)

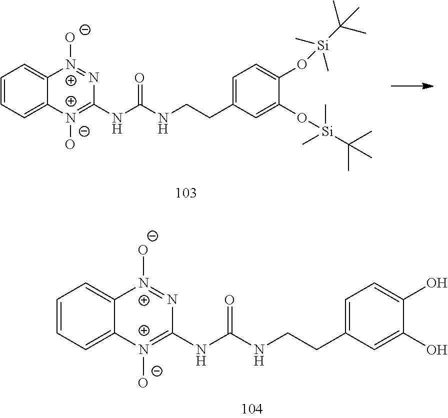 [SMILES: CC(C)(C)[Si](C)(C)Oc1ccc(CCNC(=O)Nc2n[n+]([O-])c3ccccc3[n+]2[O-])cc1O[Si](C)(C)C(C)(C)C ; O=C(NCCc1ccc(O)c(O)c1)Nc1n[n+]([O-])c2ccccc2[n+]1[O-]]

Di-TBDMS protected TPZ-DOPA 103 (200 mg, 0.56 mmol, 1.0 eq.) was dissolved in isopropyl alcohol (16 mL) under an argon atmosphere. 1 M aqueous HCl (4 mL) was then added and the reaction mixture was left to stir at room temperature for 48 hours. The precipitate formed was isolated by centrifugation and washed thoroughly with isopropyl alcohol, and then with hexane to yield 97.8 mg of TPZ-DOPA 104 as a yellow solid after drying under vacuum. (Yield: 80%). 1H NMR (400 MHz, DMSO-d₆), δ ppm: 2.60 (t, 2H), 3.30-3.35 (m, 2H), 6.49 (dd, 1H), 6.61-6.67 (m, 2H), 7.61 (t, 1H), 7.76 (ddd, 1H), 8.03 (ddd, 1H), 8.26 (d, 1H), 8.30 (d, 1H), 8.68 (s, 1H), 8.78 (s, 1H), 9.98 (br. s, 1H); ¹³C NMR (10 MHz, DMSO-d₆), δ ppm: 35.0, 41.8, 116.2, 116.6, 118.5, 120.2, 121.7, 130.4, 130.7, 132.7, 136.9, 138.6, 144.1, 145.6, 146.5, 151.6; IR (cm⁻¹) (solid): 3475, 3333, 3082, 2938, 1664, 1593, 1519, 1488, 1415, 1335, 1293, 1268, 1188, 1119, 1094, 957; MS m/z (ES+) 358.1 [MH+], 380.1 [MNa⁺], m/z (ES−) 356.1 [MH⁻]; HR-MS m/z calculated for C₁₆H₁₅N₅O₅Na [MNa⁺]=380.097; m/z (HR-ES+) 380.097 [MNa⁺].

### Preparation of HAB-TPZ-DOPA

The HAB-TPZ-DOPA macromolecular pro-drug was prepared by procedures analogous to those used for the preparation of HABQ and HABP, as described in Example 1, replacing TPZ-DOPA 104 for quercetin/piceatannol.

### Tumour Cell Viability Assays

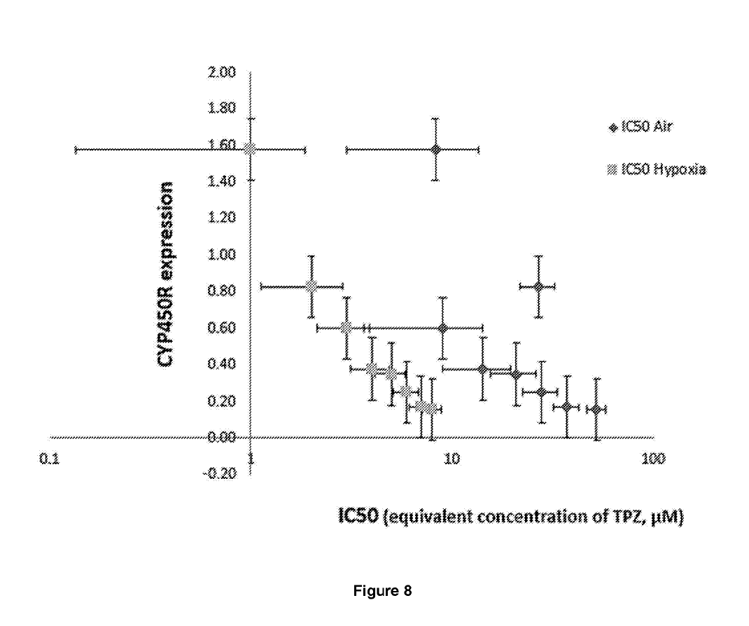

A number of human tumoral cell lines were screened in terms of the production of cytochrome P450 reductase (Western blots); the toxicity of HAB-TPZ-DOPA was evaluated by screening the viability of the cells after 3 h exposure to the conjugate as a function of the molar concentration of TPZ groups, and expressing the corresponding IC50 values. The results are shown in FIG. 8. The conjugate HAB-TPZ-DOPA demonstrated clear hypoxia-activatable behaviour, with cytotoxicity of the conjugate increased under hypoxic conditions compared with normal (air) conditions, as seen by lower hypoxic IC50 values. The conjugate cytotoxicity also increased with increased expression levels of P450 reductase.

In comparative cytotoxicity experiments, a panel of human cancer cell lines were subjected to 3 h exposure to TPZ, TPZ-DOPA, or HAB-TPZ-DOPA and assessed for cell survival at 72 h under both normoxic (air) and hypoxic (0.1% 02) conditions. IC50 values (μM) were determined from n=3 experiments and are presented in Table 1.

TABLE 1

| | TPZ | | TPZ-DOPA | | HAB-TPZ-DOPA | |
|---|---|---|---|---|---|---|
| Cell Line | Air | 0.1% O₂ | Air | 0.1% O₂ | Air | 0.1% O₂ |
| A549 | 585.0 ± 26.3 | 81.3 ± 7.2 | 83.6 ± 3.7 | 42.3 ± 1.2 | 77.0 ± 1.5 | 56.0 ± 1.2 |
| Calu-3 | 241.7 ± 6.0 | 71.7 ± 4.4 | 20.6 ± 1.1 | 16.7 ± 0.9 | 42.3 ± 2.8 | 28.0 ± 4.3 |
| Calu-6 | 340.0 ± 11.5 | 75.0 ± 8.6 | 26.7 ± 4.8 | 8.3 ± 0.9 | 33.0 ± 3.0 | 11.3 ± 1.2 |
| HepG2 | 227.5 ± 40.1 | 13 ± 4.7 | 13.75 ± 2.8 | 3.7 ± 0.7 | 18.3 ± 1.9 | 3.7 ± 0.9 |
| Hep3B2 | >500 | 31.2 ± 2.3 | 21.8 ± 2.7 | 4.8 ± 0.8 | 30 ± 2.4 | 5.0 ± 0.8 |
| SNU-423 | >500 | 40 ± 5.8 | 22.5 ± 6.4 | 6.0 ± 1.0 | 31.8 ± 6.4 | 7.0 ± 0.3 |
| SKBr3 | 217.5 ± 3.5 | 6.3 ± 1.3 | 17.7 ± 1.5 | 13 ± 1.3 | 21.6 ± 1.5 | 4.4 ± 3.1 |
| MDA-MB-468 | 232.5 ± 35 | 7.5 ± 4.1 | 17.5 ± 5 | 8.2 ± 2.6 | 8.3 ± 2 | 3.3 ± 1 |
| T47D | 271 ± 36.4 | 17.1 ± 1.0 | 35.5 ± 6.4 | 18 ± 3.7 | 21 ± 23.2 | 4.2 ± 2.6 |
| MCF-7 | 310 ± 18.3 | 20.0 ± 2.0 | 23.1 ± 5.5 | 15.4 ± 7.09 | 17.8 ± 6.5 | 4.3 ± 3.9 |
| MDA-MB-231 | 341 ± 37.5 | 30 ± 5 | 23.1 ± 5.3 | 9.0 ± 1.4 | 62.2 ± 14.12 | 4.2 ± 2.3 |
| FaDu | 383.3 ± 104.1 | 23.3 ± 5.4 | 18 ± 2 | 6.5 ± 0.9 | 37.7 ± 10.5 | 4.3 ± 0.3 |
| MIA PACA-2 | 270 ± 10 | 15.3 ± 2.5 | 28 ± 3.7 | 9.6 ± 3.4 | 30.8 ± 6.7 | 4.7 ± 1.1 |
| CAL-27 | 318 ± 50.2 | 10 ± 2 | 18.7 ± 3.1 | 7.5 ± 4.4 | 7.8 ± 2.4 | 2.5 ± 1 |

Table 1 shows that the cancer cell viability under hypoxia was always considerably lower than that under normoxia. HAB-TPZ-DOPA had increased overall toxicity compared to TPZ, although TPZ showed a larger difference between hypoxic and normoxic cytotoxicity.

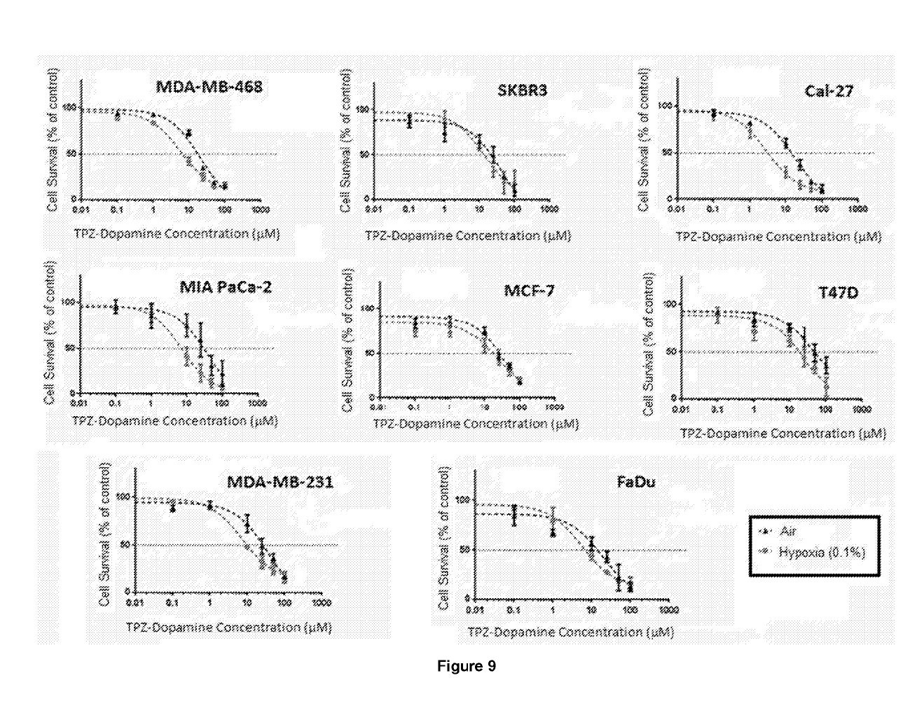

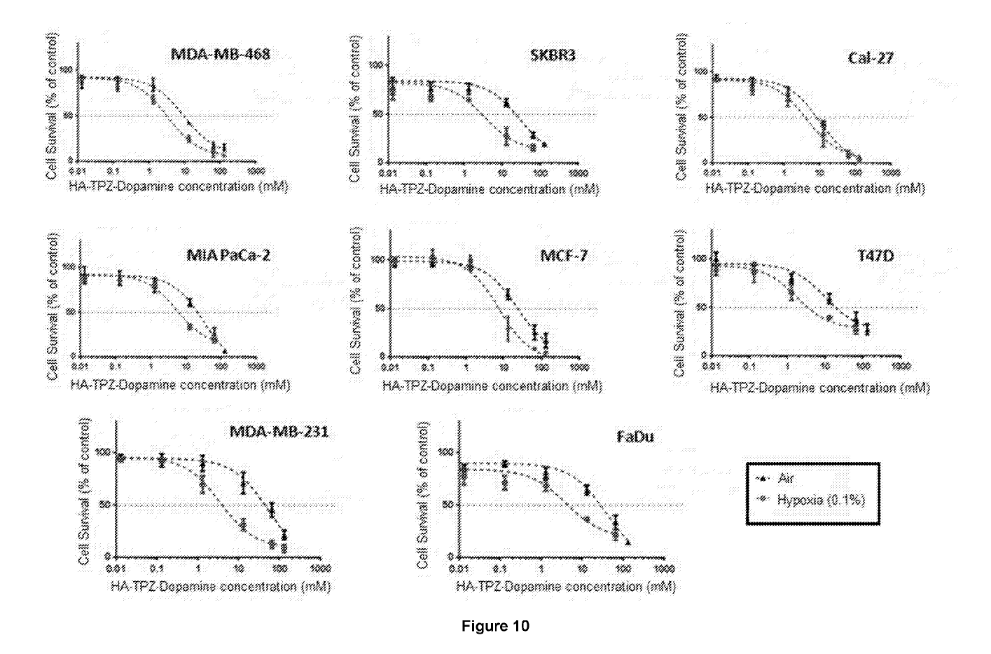

72 h viability data for eight of the cancer cell lines (MDA-MB-468, SKBr3, Cal-27, MIA PaCa-2, MCF-7, T47D, MDA-MB-231 and FaDu) are also shown in FIG. 9 and FIG. 10 after 3 h exposure to TPZ-DOPA and HAB-TPZ-DOPA respectively. In FIG. 9 it can be seen that the viability is lower under hypoxia than that under normoxia. In FIG. 10, for the HAB-TPZ-DOPA conjugate the hypoxic viability was always considerably lower than that under normoxia, and with a greater hypoxia/normoxia difference in most cases than for TPZ-DOPA in FIG. 9.

### Example 8—Measurement of the Toxicity of Curcumin and a Curcumin-HAB Conjugate to Breast Cancer Cells

### Method

MDA-MB-231 breast cancer cells were cultured in RPMI-1640 (Gibco) containing 10% FBS, 1% Pen-Strep, L-Glutamine 2 mM (medium) at 37° C. in a humidified 5% CO₂ atmosphere. To determine the toxicity of curcumin and a curcumin-HAB conjugate, cells were seeded in 96-well plates at a density of 1,000 cells per well in medium and left to attach for 24 hours. Medium was then removed and replaced with medium containing various concentrations of curcumin or the curcumin-HA conjugate. The cells were then incubated for 3 hours at 37° C. in air plus 5% CO₂. After this time, drug containing medium was removed, replaced with fresh medium and cells incubated for a further 96 hours. Thereafter cell survival was determined using the a modified MTT [3-(4,5-dimethyldiazol-2-yl)-2,5-diphenyltetrazolium bromide] method according to the manufacturer's instructions (Dojindo Molecular Technologies Inc., Rockville, Md.). Briefly, 50 μl of MTT solution (0.5 mg/ml in cell culture medium) was added to each well. After incubation for a further 4 h at 37° C. the medium was removed from each well and replaced with 200 μl of DMSO to dissolve the formazan crystals. The Absorbance in each well was then read at a wavelength of 450 nm using a Tecan Infinite M200 plate-reader using I-control software. The relative cell viability (%) was calculated by the formula [A]ₜₑₛₜ/[A]_{control}×100, where “[A]ₜₑₛₜ” is the absorbance of the test sample, and “[A]_{control}” is the absorbance of the control cells incubated solely with culture medium.

### Results

In these experiments, the maximum cell kill that could be achieved with curcumin was 40%. This was obtained using a 40 μM concentration of curcumin. In contrast, the concentration of curcumin contained in the curcumin-HAB conjugate needed to give this level of cell kill was 5 μM. This 8-fold reduction is consistent with enhanced delivery of curcumin via conjugation with HAB.

### REFERENCES

1 Yuan, Z. P. et al. Liposomal quercetin efficiently suppresses growth of solid tumors in murine models. Clinical Cancer Research 12, 3193-3199 (2006).
2 Fang, F., Li, J.-M., Pan, Q.-H. & Huang, W.-D. Determination of red wine flavonoids by hplc and effect of aging. Food Chemistry 101, 428-433 (2007).
3 Sixou, S. et al. Intracellular oligonucleotide hybridization detected by fluorescence resonance energy-transfer (fret). Nucleic Acids Research 22, 662-668 (1994).
4 Yang, J. H. et al. Inhibition of lung cancer cell growth by quercetin glucuronides via g(2)/m arrest and induction of apoptosis. Drug Metabolism and Disposition 34, 296-304 (2006).
5 Indap, M. A., Radhika, S., Motiwale, L. & Rao, K. V. K. Quercetin: Antitumor activity and pharmacological manipulations for increased therapeutic gains. Indian Journal of Pharmaceutical Sciences 68, 465-469 (2006).
6 Kubo, I., Nitoda, T. & Nihei, K.-i. Effects of quercetin on mushroom tyrosinase and b16-f10 melanoma cells. Molecules 12, 1045-1056 (2007).
7 Daker, M., Ahmad, M. & Khoo, A. S. B. Quercetin-induced inhibition and synergistic activity with cisplatin—a chemotherapeutic strategy for nasopharyngeal carcinoma cells. Cancer Cell International 12 (2012).
8 Potter, G. A. et al. The cancer preventative agent resveratrol is converted to the anticancer agent piceatannol by the cytochrome p450 enzyme cypibi. British Journal of Cancer 86, 774-778 (2002).
9 Wieder, T. et al. Piceatannol, a hydroxylated analog of the chemopreventive agent resveratrol, is a potent inducer of apoptosis in the lymphoma cell line bjab and in primary, leukemic lymphoblasts. Leukemia 15, 1735-1742 (2001).
10 Bieger, J. et al. Tissue distribution of quercetin in pigs after long-term dietary supplementation. Journal of Nutrition 138, 1417-1420 (2008).
11 de Boer, V. C. J. et al. Tissue distribution of quercetin in rats and pigs. Journal of Nutrition 135, 1718-1725 (2005).
12 Fraser, J. R. E., Appelgren, L. E. & Laurent, T. C. Tissue uptake of circulating hyaluronic-acid—a whole-body autoradiographic study. Cell and Tissue Research 233, 285-293 (1983).
13 Fraser, J. R. E., Laurent, T. C., Pertoft, H. & Baxter, E. Plasma-clearance, tissue distribution and metabolism of hyaluronic-acid injected intravenously in the rabbit. Biochemical Journal 200, 415-424 (1981).
14 Gustafson, S., Bjorkman, T. & Westlin, J. E. Labeling of high-molecular-weight hyaluronan with i-125 tyrosine—studies in-vitro and in-vivo in the rat. Glycoconjugate Journal 11, 608-613 (1994).
15 El-Dakdouki, M. H. et al. Assessing the in vivo efficacy of doxorubicin loaded hyaluronan nanoparticles. Acs Applied Materials & Interfaces 6, 697-705 (2014).
16 Ganesh, S., Iyer, A. K., Gattacceca, F., Morrissey, D. V. & Amiji, M. M. In vivo biodistribution of sirna and cisplatin administered using cd44-targeted hyaluronic acid nanoparticles. Journal of Controlled Release 172, 699-706 (2013).
17 Yoshida, T. et al. Antiandrogen bicalutamide promotes tumor growth in a novel androgen-dependent prostate cancer xenograft model derived from a bicalutamide-treated patient. Cancer Research 65, 9611-9616 (2005).

1. A polymer-drug conjugate, or a pharmaceutically acceptable salt or solvate thereof, for use in the treatment of cancer,
wherein the polymer is a modified hyaluronic acid derivative and the drug is an anticancer compound, and wherein the anticancer compound is covalently linked to the modified hyaluronic acid derivative by a pH labile boron-containing linkage.

2. A polymer-drug conjugate, or a pharmaceutically acceptable salt or solvate thereof, for use in the treatment of cancer according to claim 1, wherein the modified hyaluronic acid derivative is a hyaluronic acid polymer in which a proportion of the hyaluronic acid monomer units have been chemically modified to include one or more pendant boronate acid-containing moieties which react with the anticancer drug to form a pH labile boron-containing linkage.

3. A polymer-drug conjugate, or a pharmaceutically acceptable salt or solvate thereof, for use in the treatment of cancer according to claim 2, wherein 1 to 30% of the monomeric units present in the HA polymer backbone comprise pendant boronic acid-containing moieties.

4. A polymer-drug conjugate, or a pharmaceutically acceptable salt or solvate thereof, for use in the treatment of cancer according to claim 2, wherein 5 to 20% of the monomeric units present in the HA polymer backbone comprise pendant boronic acid-containing moieties.

5. A polymer-drug conjugate, or a pharmaceutically acceptable salt or solvate thereof, for use in the treatment of cancer according to claim 1, wherein the modified hyaluronic acid derivative is a soluble HA polymer that has a molecular weight within the range of 100 kDa to 1 MDa.

6. A polymer-drug conjugate, or a pharmaceutically acceptable salt or solvate thereof, for use in the treatment of cancer according to claim 1, wherein the modified hyaluronic acid derivative is a soluble HA polymer that has a molecular weight within the range of 300 kDa to 800 kDa.

7. A polymer-drug conjugate, or a pharmaceutically acceptable salt or solvate thereof, for use in the treatment of cancer according to claim 2, wherein the pendant boronic acid-containing moiety is bound to the carboxy group of a HA monomer by an amide linkage.

8. A polymer-drug conjugate, or a pharmaceutically acceptable salt or solvate thereof, for use in the treatment of cancer according to claim 2, wherein the pendant boronate-containing moiety has the general structural formula II shown below: 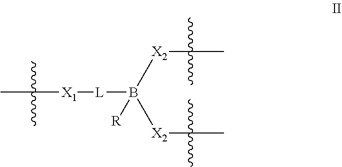
wherein:
L is linking group;
R is absent or selected from: A) an OH group; or B) a group —Xᵣ-Qᵣ- formed by the association of a substituent group of the formula -QᵣXᵣH present on either the modified HA polymer or the drug molecule with the boron atom, wherein Xᵣ is a heteroatom linker selected from —O—, —NR_{z}— (where R_{z} is H or (1-4C)alkyl) or —S— and Qᵣ is the remainder of the substituent group present on either the modified HA polymer or the drug molecule;
X₁ is a functional group (e.g. —NH— or —O—) that connects the linking group L to the —C(═O)— of the carboxylic acid group present in the hyaluronic acid monomeric unit; and
X₂ is O or NH.

9. A polymer-drug conjugate, or a pharmaceutically acceptable salt or solvate thereof, for use in the treatment of cancer according to claim 8, wherein the pendant boronate-containing moiety has the general structural formula IIa shown below: 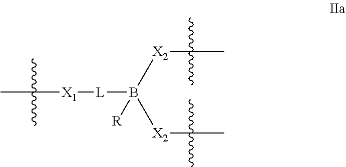
wherein:
L is an optionally substituted phenyl group;
R is as defined in claim 8;
X₁ is —NH—; and
X₂ is O or NH.

10. A polymer-drug conjugate, or a pharmaceutically acceptable salt or solvate thereof, for use in the treatment of cancer according to claim 9, wherein the pendant boronate-containing moiety has the general structural formula IIc shown below: 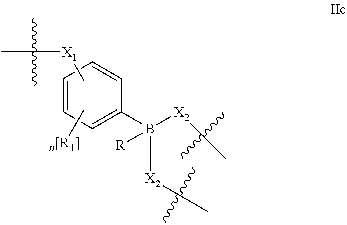
wherein:
X is —NH—;
R is as defined in claim 8;
R₁ is amino;
n is 0 or 1; and
X₂ is O or NH.

11. A polymer-drug conjugate, or a pharmaceutically acceptable salt or solvate thereof, for use in the treatment of cancer according to claim 1, wherein the anticancer drug comprises a catechol moiety, or is linked to a linker group that comprises a catechol moiety, that binds to a pendant boronic acid group of the modified HA polymer to form a pH-labile cyclic boronate ester linkage.

12. A polymer-drug conjugate, or a pharmaceutically acceptable salt or solvate thereof, for use in the treatment of cancer according to claim 11, wherein the anticancer drug is selected from quercetin, tannic acid, piceatannol, taxifolin, catechin, curcumin, or tirapazamine.

13. A pharmaceutical composition for use in the treatment of cancer comprising a polymer-drug conjugate according to claim 1, or a pharmaceutically acceptable salt or solvate thereof, and one or more pharmaceutically acceptable excipients.

14.-15. (canceled)

16. A method of treating cancer in a patient in need of such treatment, said method comprising administering to said patient a therapeutically effective amount of a polymer-drug conjugate according to claim 1.

17. A method of treating solid tumors in a patient in need of such treatment, said method comprising administering to said patient a therapeutically effective amount of a polymer-drug conjugate according to claim 1.
